(19) United States
(12) Patent Application Publication
(10) Pub. No.: US 2008/0237028 A1
(43) Pub. Date: Oct. 2, 2008

(54) Nucleation in liquid, methods of use thereof and methods of generation thereof

(75) Inventor: Hanoch Kislev, Zichron Yaakov (IL)

Correspondence Address: STAAS & HALSEY LLP, SUITE 700, 1201 NEW YORK AVENUE, N.W., WASHINGTON, DC, 20005, US

(73) Assignee: Hanoch KISLEV, Zichron Yaakov (IL)

(21) Appl. No.: 11/896,751

(22) Filed: Sep. 5, 2007

Related U.S. Application Data
(60) Provisional application No. 60/842,597, filed on Sep. 5, 2006.
(60) Provisional application No. 60/851,374, filed on Oct. 13, 2006.

(51) Int. Cl. B01J 19/12; B01J 19/10; A61K 9/14; C12N 13/00; A61P 27/00

(52) U.S. Cl. 204/157.15; 422/128; 424/489; 424/427; 435/173.1; 977/742

(57) ABSTRACT

A method and composition for generation of a microbubble from a nanoparticle through a non-thermal method, preferably featuring nucleation.

### FIELD OF THE INVENTION

The present invention relates to nucleation bubbles, and methods of preparation and use thereof, and in particular, to generation of nucleation bubbles through non-thermal interaction of electromagnetic radiation with nanoparticles.

### BACKGROUND OF THE INVENTION

There are many medical applications that would benefit from the generation of microbubbles in specific body regions [1,2]. At present the mechanism for extracorporeal generation of nucleation bubble in vivo is HIFU. Another possibility is based on administering encapsulated nanobubbles with suitable ligands to desired regions [3].

As described by Kislev in WO 2006 051542, it is possible to generate nucleation bubbles by exposing the nanoparticles to pulsed light and heat them to several hundred degrees C. needed for generating vapor nucleation bubbles.

However, the penetration depth of light, including infrared light, is limited. The use of microwave electromagnetic radiation has significant advantage over photonic radiation due to its deep penetration into typical tissue (2-5 cm for 1 GHz radiation). Microwave radiation, and especially in the low GHz, can penetrate through the body [4]. There are nanoparticles which absorb microwave energy. The interaction of certain dissolved compounds with microwaves to generate microbubbles has been described by Kiel et al. [5].

Unfortunately, the attainable power density of pulsed microwave fields are orders of magnitude lower than laser beams, such as for example 100 kW/cm² due to gas or liquid breakdown problems within the microwave source, as the amount of energy required for vapor nucleation and for generation of microbubbles exceeded that which could be withstood by the apparatus itself. Therefore practical microwave power densities are far below those required for heating nanoparticles to several hundred degrees C. which is required for generating vapor nucleation bubbles around absorbing nanoparticles.

### SUMMARY OF THE INVENTION

The background art does not teach or suggest a method for generating microbubbles through nucleation according to a non-thermal method.

The present invention overcomes these drawbacks of the background by providing compositions and methods for non-thermal generation of nucleation bubbles.

According to some embodiments of the present invention, there is provided a non-thermal method of generating microbubbles by using nanoparticles.

According to other embodiments of the present invention, the methods used herein may optionally be applied to treat biofilms, optionally and preferably in combination with another therapy, more preferably comprising at least one antibiotic.

According to yet other embodiments of the present invention, the methods used herein may optionally be applied to localized and/or controlled and/or directed drug release. Compositions for such drug release, preferably comprising particles which comprise nanoparticles as described herein, are also preferably provided according to some embodiments.

According to still other embodiments of the present invention, the methods used herein may optionally be applied to induction of therapeutic embolization, for example to occlude a blood vessel in a diseased tissue. The diseased tissue may optionally comprise a tissue having a disease characterized by angiogenesis, including but not limited to, cancer, excessive bleeding, bleeding at an inappropriate location and rheumatoid arthritis. Compositions for such embolization, preferably comprising particles which comprise nanoparticles as described herein, are also preferably provided according to some embodiments.

According to still other embodiments of the present invention, the methods used herein may optionally be applied to diagnostic imaging.

According to some embodiments of the present invention, there is provided a method for generating a nucleation bubble in a non-thermal process, comprising: Providing a nanoparticle in a liquid environment; and Applying electromagnetic radiation to the nanoparticle to induce formation of a nucleation bubble.

Preferably, the electromagnetic radiation comprises microwave radiation. More preferably, the nanoparticle induces local electromagnetic radiation whose electric field magnitude is at least five times ambient electromagnetic field.

The method optionally and more preferably further comprises applying ultrasound to the nanoparticles. Most preferably, the ultrasound is applied to grow the nucleation bubble to form a microbubble.

Optionally, the electromagnetic radiation comprises microwave radiation of a frequency from about 20 MHz to about 1000 GHz. Preferably, the frequency is from about 100 MHz to about 3 GHz. More preferably, a source pulse width of the microwave radiation is from about 10 nanosecond to about 30 milliseconds. Most preferably, the source pulse width is from about 0.01 to about 10 microsecond.

Preferably, an average microwave power density is from about 0.1 kW/cm2 to about 1 MW/cm2. More preferably, the power density is from about 1 kW/cm2 to about 100 kW/cm2.

Preferably, the microwave radiation has a mode selected from the group consisting of single pulse mode, pulse train mode, or repeated sequence mode.

Optionally the method further comprises applying ultrasound having an ultrasound source frequency of from about 20 kHz to about 10 MHz.

Preferably, the ultrasound source frequency is from about 0.5 to about 10 MHz. More preferably, the ultrasound source frequency is from about 0.75 to about 3 MHz.

Optionally, an energy level of the ultrasound is from about 0.5 Watt (W) per square centimeter (cm.sup.2) to about 20 W/cm.sup.2. Preferably, the energy level of the ultrasound is from about 0.5 to about 2.5 W/cm.sup.2.

Optionally the method further comprises synchronizing applying the ultrasound radiation and the microwave radiation. Optionally the method further comprises providing nanoparticles to an object to be treated. Preferably, the object to be treated comprises one or more of tissue, a body of a subject, a non-living surface, a biofilm, micro-organisms, a blood vessel and a tumor.

Optionally the liquid further comprises one or more of water, aqueous solution, non-aqueous solution, gel, semi-solid, suspension, dispersion, or membrane, or a combination thereof. Also optionally the nanoparticles comprise conductive material in the microwave frequencies. Preferably the conductive material comprises one or more of metal, metal alloy, carbon, and semiconductors.

Optionally, a nanoparticle interaction cross section at microwave radiation frequencies is from about 0.05 to about 0.5 of their geometric cross section.

Also optionally a size of the nanoparticle is determined according to one or more characteristics selected from the group consisting of shape, structure, materials, operation conditions and requirements of a specific application, or a combination thereof. Optionally, a shape of the nanoparticle is selected from the group consisting of nanotubes, high aspect ratio rods or ellipsoids, and nanoshells.

Preferably the nanoparticle further comprises one or more local nanometer sized structures for enhancing a local electric field in their vicinity. More preferably, the nanoparticle comprises a nanotube selected from the group consisting of carbon nanotubes (CNT), Boron nitride nanotubes, BCN nanotubes, in which some carbon atoms were replaced by nitrogen and boron atoms (BCNT) silicone carbide nanotubes, bundles of single-wall carbon nanotubes, multi-wall carbon nanotubes, buckytubes, fullerene tubes, carbon fibrils, carbon nanotubules, carbon nanofibers, and combination thereof. Most preferably, the nanotube is a multi walled nanotube comprising nanoscrolls, nanofibrils, nanovessels, nanocontainers, and combinations thereof.

Optionally, the nanoparticle comprises at least one site for promoting the accumulation of gas molecules generated by exposing the nanoparticle to microwave radiation. Preferably, the site comprises a discontinuity suitable for accumulation of gas molecules as an attached nanobubble. More preferably, the site has a surface morphology selected from the group consisting of a crack, a depression, a linear edge, a pointed edge, a boundary between hydrophobic and hydrophilic materials, and a boundary between different surface characteristics.

According to some embodiments of the present invention, there is provided a method for generating a microbubble in a non-thermal process, comprising: Providing a nanoparticle in a liquid environment; Applying electromagnetic radiation to the nanoparticle to induce formation of a gas nucleation bubble; and Applying ultrasound to form a microbubble from the gas nucleation bubble. Optionally the method further comprises Modulating the microbubble through application of the ultrasound.

According to some embodiments of the present invention, there is provided a method for generating a microbubble, comprising: Providing a nanoparticle in a liquid environment; Applying electromagnetic radiation to the nanoparticle to induce formation of reactive species molecules; Generating a gas nucleation bubble from a reaction of the reactive species and the liquid environment; and Forming a microbubble from the gas nucleation bubble.

According to some embodiments of the present invention, there is provided a method for generating a microbubble, comprising: Providing a nanoparticle in a liquid environment; Applying microwave radiation to the nanoparticle to induce formation of a gas nucleation bubble; Applying ultrasound radiation to the gas nucleation bubble to form a microbubble; and Increasing a size of the microbubble through continued application of the ultrasound radiation. Preferably, the microwave radiation and the ultrasound radiation are applied in an overlapping manner. More preferably the microwave radiation and the ultrasound radiation are applied simultaneously.

According to some embodiments of the present invention, there is provided a composition for inducing formation of a microbubble upon application of a non-thermal process, comprising a nanoparticle having a surface featuring at least one characteristic for accumulation of gas molecules, wherein the gas molecules form a nucleation seed for the microbubble.

According to some embodiments of the present invention, there is provided a nanoparticle, comprising a solid portion and/or a coating for responding as a cohesive whole to electromagnetic radiation in a liquid environment for generating microbubbles in a non-thermal process. Preferably the nanoparticle comprises a coating featuring biological functionalization. More preferably, the coating is selected from the group consisting of a coating being covalently bound to the surface of the nanoparticle and a coating physically adhering to the surface of the nanoparticle. Most preferably the coating comprises a material fro providing one or more of the following functions: stabilize the absorbing nanoparticles in aqueous suspension to prevent their aggregation; prevent uptake of absorbing nanoparticles by the immune system if administered to a body of a subject; serve as an intermediate layer for attachment of targeting ligands; enhance nanoparticle transport through blood vessels and interstitial regions; maintain the capability of nanoparticles to effectively generate nucleation bubbles.

Optionally the coating comprises one or more of a surfactant, linker, spacer, targeting ligand and encasing ligand. Preferably the surfactant comprises one or more of polymeric or non-polymeric surfactants. More preferably the polymeric surfactant comprises a block copolymer. Most preferably the block copolymer has a block comprising poly(ethylene glycol) (PEG).

Optionally the surfactant comprises a protein, modified protein or other biological molecule.

Also optionally the coating extends the circulation lifetime of the nanoparticle in a body of a subject by minimizing uptake by the immune system when administered to the body of the subject. Optionally the coating comprises one or more reactive functional groups. Preferably, the coating further comprises a linker and/or a spacer. More preferably, the coating further comprises a targeting ligand.

Optionally, a size of the nanoparticle is selected to control a biological property selected from the group consisting of the bio-distribution, penetration through vasculature and interstitial volume, and the blood clearance rate, or a combination thereof. Preferably, the size is from about 10 to about 1000 nm. More preferably the size is from about 10 to about 100 nm.

According to some embodiments of the present invention, there is provided a composition comprising a nanoparticle as described herein in a liposome. Preferably, the composition further comprises a biologically active agent for being contained in the liposome.

According to some embodiments of the present invention, there is provided a system for inducing a microbubble in a non-thermal process, comprising: a. a source of microwave radiation; b. a source of ultrasound radiation; c. a guide for the microwave radiation and the ultrasound radiation; and d. a nanoparticle in a liquid environment for receiving the microwave radiation and the ultrasound radiation, and for generating the microbubble.

According to some embodiments of the present invention, there is provided a method for biofilm treatment, comprising: generating a microbubble in a non-thermal process as described herein. Optionally the method further comprises at least reducing a biological efficacy of the biofilm. Preferably the method further comprises disrupting the biofilm. More preferably the method further comprises treating the biofilm with an additional bioactive substance. Most preferably, the bioactive substance is selected from the group consisting of an antibiotic, immune system stimulant and a bacteriophage.

Optionally, the biofilm is present in a tissue of a subject. Optionally, the biofilm is present on a non-living surface. Preferably, the non-living surface comprises an implant. More preferably, the non-living surface comprises a coating layer suitable for fixating nanoparticles, on which fixated absorbing nanoparticles are present. More preferably, the absorbing nanoparticles are arranged in clusters and the clusters comprise between 5 and 50 nanoparticles each. Most preferably, an average inter-nanoparticle distance ranges from about 0.1 to about 3 microns.

Also most preferably a structure of the absorbing nanoparticle anchors the nanoparticle to the coating and provides generation of nucleation at its exposed section. Optionally, the coating comprises a material having thermal insulation properties. Preferably, the material comprises a ceramic material. Optionally, the nanoparticles are integrated into the coating material and the coating material is porous.

According to some embodiments of the present invention, there is provided a composition for delivery of a bioactive agent comprising: a particle comprising a bioactive composition, volatile liquid, and absorbing nanoparticles operable for inducing delivery of the bioactive agent when exposed to suitable electromagnetic and ultrasound radiation. Preferably, a size of the particle is from about 200 nm to about 10 micron. More preferably, the particle comprises a shell, wherein a thickness of the particle shell is determined according to mechanical properties of the shell and according to a release mechanism for the bioactive agent. Most preferably, the thickness is from about 25 nm to about 1000 nm. Optionally, the shell comprises a lipid. Preferably, the particle comprises a liposome. More preferably, the nanoparticle is encapsulated in the aqueous interior of the liposome, interspersed within the lipid bilayer of the liposome, attached to the liposome via a linking molecule that is associated with both the liposome and the absorbing nanoparticle, entrapped in the liposome, or complexed with the liposome. Most preferably, the composition further comprises a hydrophilic agent for coating the liposome.

Optionally the composition further comprises an emulsion for containing the lipid encapsulated particles. Preferably the emulsion comprises a derivatized natural or synthetic phospholipid, a fatty acid, cholesterol, lipolipid, sphingomyelin, tocopherol, glucolipid, stearylamine, cardiolipin, a lipid with ether or ester linked fatty acids or a polymerized lipid.

According to some embodiments of the present invention, there is provided a method for localized delivery of a bioactive composition from a particle, comprising: delivering a particle comprising bioactive composition, absorbing nanoparticles and volatile composition to cells or tissue; and exposing the particle to simultaneous electromagnetic radiation beam and ultrasound radiation to induce release of the bioactive composition.

Preferably the method further comprises: generating a microbubble within the particle sufficient to evaporate at least a fraction of the volatile composition; and breaching the particle due to enhanced internal pressure, thereby causing release of its bioactive content to the cells or tissue. Optionally electromagnetic radiation and ultrasound energy are below an evaporative rupture threshold.

According to some embodiments of the present invention, there is provided a method for localized delivery of therapeutic or bioactive composition from a particle, comprising: delivering a particle comprising bioactive composition, absorbing nanoparticles and pro-permeable membrane wall and an attached ligand suitable for attachment to a targeted cell, to the eye; contacting the particle to selected ocular target cells using a suitable ligand; and exposing the particle simultaneous electromagnetic radiation beam and ultrasound radiation.

Optionally the method further comprises generating a microbubble near inner wall of the particle; and inducing permeability of the membrane shell due to pulsation of the microbubble, in turn enabling enhanced transport of the bioactive compositions from the particle to the targeted cells or tissue.

According to some embodiments of the present invention, there is provided a method for localized embolization of a blood vessel, comprising generating a microbubble in a non-thermal process as described herein; and occluding the blood vessel with the microbubble.

Preferably, the generating the microbubble comprises applying microwave radiation and ultrasound radiation to a nanoparticle. The method may optionally be applied for treating an angiogenesis-dependent disease. Preferably the angiogenesis-dependent disease comprises cancer.

Optionally the method comprises administering a particle to the blood vessel, the particle comprising a volatile liquid and absorbing nanoparticles. Preferably an amount of the volatile liquid is selected to provide a gas bubble volume of from about 0.5 to about 3 times a diameter of the blood vessel to be occluded, cubed.

Optionally the method comprises administering at least one particle comprising volatile liquid suitable for generating a gas bubble to the blood vessel; exposing the vasculature system(s) to simultaneous electromagnetic radiation and ultrasound radiation so as to generate nucleation bubble within the particle; continuing exposure of the particle to ultrasound for causing release of the volatile liquid from particle as vapor; and generating gas bubbles within the blood vessel, such that the blood vessel is effectively occluded.

According to some embodiments of the present invention, there is provided a method for imaging diagnostics in a subject, comprising generating a microbubble in a non-thermal process as described herein; and imaging at least a portion of the subject from the microbubble.

According to some embodiments of the present invention, there is provided a method for inducing hyperthermia in a subject, comprising generating a microbubble in a non-thermal process as described herein.

### DEFINITIONS

As used herein, “electromagnetic radiation” is defined as radiation having an electric field and a magnetic field propagating at right angles to one another. As used herein “microwave” is electromagnetic radiation selected from the group: Terahertz radiation, millimeter waves, microwaves and Very High Frequency radio-frequency radiation. As used herein “optical radiation” is electromagnetic radiation selected from the group “far infra-red, near infra-red, visible, and ultra violet radiation.

The term “absorbing nanoparticle” refers to either to single nanoparticles, or nanoparticles assembled as clusters or agglomerates, exhibiting enhance absorption of specific portion of the electromagnetic spectrum compared to randomly shaped nanoparticles of the same size.

The term “microbubble” refers to a cavity in liquid comprising a mixture of non-condensable gas and vapor whose size exceeds 1 micron and has a significant interaction cross section with ultrasound radiation in the low MHz frequency range. Microbubbles may optionally be stabilized through shell encapsulation.

The term “nanobubble” refers to a cavity in liquid comprising a mixture of non-condensable gas and vapor whose size is below 1 micron. Nanobubbles may be short lived and are optionally stabilized through shell encapsulation, or optionally may be attached to a solid surface.

The term “nucleation site” or “nucleation bubble” stands for a volume with a finite size measured in nanometers, which upon exposure to suitable ultrasound radiation can be temporarily stabilized or evolved into a larger bubble.

The term “thermal nucleation” refers to a process during which an absorbing nanoparticle immersed in a liquid is exposed to suitable electromagnetic radiation, heats up above the local boiling point of the liquid and in turn vaporizes a sufficient volume of the liquid to serve as a nucleation site.

The term “non-thermal nucleation” refers to a process during which an absorbing nanoparticle immersed in a liquid is exposed to suitable electromagnetic radiation, and in turn generates a nucleation bubble, either by generation of non-condensable gas molecules or collecting non-condensable gas molecules from the liquid sufficiently to serve as a nucleation site.

As used herein, “cluster” is defined as a plurality of nanoparticles spread on a surface of a tissue whose size is measured in a few microns. The term “agglomerate” is defined as a plurality of nanoparticles agglomerated in a 3-dimensional structure.

The term “Mechanical Index” or “MI” is defined by the equation:

$$MI = \frac{{P}_{r\_max}}{\sqrt{f}}$$

Where P.sub.r_max is the peak negative (rarefaction) ultrasound pressure in MPa and f the frequency in MHz.

As used herein the term “about” refers to ±10%.

All references, including all US patents and applications, are hereby incorporated by reference as if fully set forth herein.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates an immersed absorbing nanoparticle operable for generating a nucleation bubble by exposure to microwave generation.

FIG. 2 shows a preferred embodiment of a combined electromagnetic radiation-ultrasound radiation therapeutic method for at least reducing the efficacy of protection of a tissue-borne biofilm containing bacteria.

FIG. 3: shows the stages of an exemplary, illustrative method according to the present invention for combined treatment which enhances the anti-bacterial effect of antibiotics against bacteria within a tissue-borne biofilm.

FIG. 4: depicts an exemplary, illustrative method for combined electromagnetic radiation-ultrasound radiation therapy for at least reducing the efficacy of the protection of a biofilm on a non-tissue host surface and for combined treatment which enhances the anti-bacterial effect of antibiotics against bacteria within a tissue-borne biofilm.

FIG. 5: describes an exemplary, illustrative method for localized drug delivery from a particle comprising a cluster of absorbing nanoparticles by simultaneous exposure to ultrasound and electromagnetic radiation.

FIG. 6: describes an exemplary, illustrative method for localized drug delivery from a particle whose shell comprises absorbing nanoparticles by simultaneous exposure to ultrasound and electromagnetic radiation.

FIG. 7 shows a preferred embodiment of a combined electromagnetic radiation-ultrasound radiation therapeutic apparatus for embolizing a targeted blood vessel.

FIG. 8: shows the stages of an exemplary, illustrative method for combined treatment which leads to gas bubble formation in the targeted blood vessel.

### DETAILED DESCRIPTION OF THE INVENTION

The present invention is of a method for generating microbubbles through a non-thermal, nucleation process. Without wishing to be limited, various applications of the microbubbles are preferably encompassed within the scope of the present invention, including but not limited to treatment of biofilm and medical applications.

The following description is divided into sections for the purpose of clarity only and without wishing to be limited in any way. Section 1 describes microbubbles and methods of generation thereof. Section 2 describes various applications of microbubbles for treatment of biofilms. Section 3 describes various applications of microbubbles for local drug delivery. Section 4 describes various applications of microbubbles for embolism formation. Section 5 describes various applications of microbubbles for imaging diagnostics.

### Section 1—Microbubbles Materials Thereof and Methods of Generation Thereof

According to preferred embodiments of the present invention, there is provided a method for generating nucleation bubbles in liquid through a non-thermal process, preferably by using a plurality of nanoparticles to generate a nucleation bubble in liquid through exposure of the nanoparticles to EM (electromagnetic radiation). The non thermal process may be characterized by heating which remains below the localized boiling point of liquid at the location of nucleation. In certain aspects, the nanoparticle is minimally heated during nucleation bubble generation. As described herein, the nanoparticle is in a liquid environment, which preferably includes anything in the liquid local to the nanoparticle but which does not include the nanoparticle itself.

Each nanoparticle optionally and preferably behaves as a complete unit. Alternatively or additionally, a part of the nanoparticle may optionally behave as a complete unit as for the coating for example. By “complete unit” it is meant that the materials which comprise the nanoparticle do not respond as individual molecules for interacting with EM, but rather behave as a cohesive whole For the purpose of the present application, the EM preferably comprises microwaves or light energy, in which light energy is defined as including the infrared (near and far) and visible spectrum of light. Microwave energy is preferably used according to some embodiments of the present invention.

The absorbing nanoparticles are preferably exposed to a combination of electromagnetic radiation and ultrasound. Preferably the nucleation bubble is generated by exposing a cluster of nanoparticles to the electromagnetic radiation and ultrasound. According to other embodiments, the nucleation bubbles are evolved or “grown” by the ultrasound energy into microbubbles. In other embodiments, the microbubbles may optionally and preferably be used for treatment of tissue. In other aspects, the present invention provides controlled generation of microbubbles locally and non-invasively anywhere in the body.

As described above, various methods have been described for use in controlled generation of nucleation bubbles and microbubbles, including the use of light energy and also microwaves. However, these methods suffer from various drawbacks, such that they are not useful for the controlled generation of microbubbles in living tissue, such as in the body for example, and are particularly not useful for the controlled generation of microbubbles locally and non-invasively in the body.

The first indication regarding the potential of a non-thermal method for generating nucleation has been reported by Kiel et al., [5]. They exposed a test tube containing NaHCO3 solution doped with diazoluminomelanin (DALM) to 2-MW (estimated ˜50 kW/cm2) 5-microsecond 1.25-GHz 1-Hz pulses (0.25 W/cm2 average power). The ambient electric field based on the waveguide dimension, is estimated as 20 kV/m. During the pulses the authors observed a strong glow in the test tube and sometimes even localized thin streamer discharges. Clearly, these phenomena (bubble formation, glow, and streamers) were not observed after exposure of the same aqueous solution to a continuous low power microwave radiation.

Kiel et al., [6] used organic semiconductor molecules which were linked by a ligand to couple microwave energy to spores. A solution of DALM linked to DNA capture agents was attached to Anthrax spores and exposed to 10 millisecond 2.09 GHz microwave pulses with estimated peak power density of 15 kW/cm2 (at 10 Hz. After a few minutes, (estimated dose of 50 kJ/cm.sup.2) the spores expanded many times their original size and even ruptured. This observation seems to indicate that microbubbles were generated within the spores, although it should be noted that neither Kiel nor the literature appreciated the significance of these observations, nor were any of a nucleation process, nucleation bubble and/or their growth proposed as a cause of these observations.

The literature does not explain Kiel's observations. However there are a few indications for non-thermal microwave effects in the literature, although not related to microbubble generation. For example, Perreux and Loupy [6a] claim that microwave radiation reduces the activation energy of various reactions, thereby dramatically increases the reaction rate. One plausible explanation (although not stated by Perreux and Loupy) suggests that the microwave electric field accelerates a small portion of the reacting molecules through vibrational excitation mechanisms, thus creating a “bump on the tail” to their speed distribution. These high energy molecules react very quickly with the mating molecules and in turn increase the averaged reaction rate. The resulting accelerated reaction rate may be viewed as “reduced activation energy”. Similar to acceleration of electrons in microwave field, the acceleration level of these molecules seems to depend on the microwave electric field magnitude.

The ambient electric field within liquid environment exposed to microwave radiation can be calculated from the following equation

$$P = σ \frac{{E}_{0}^{2}}{2} \\ Where \\ σ = \frac{{ɛ}^{″}}{{ɛ}^{′}} {ɛ}^{*} = {ɛ}^{′} + i {ɛ}^{″} \qquad ( 1 )$$

Where P is the locally absorbed power density, and the liquid environment conductivity σ=0.3-0.5 1/Ω·m at 2.45 GHz. For example, the ambient peak electric field induced within a tissue whose penetration depth is 3 cm by 2.45 GHz microwave beam whose incident power density is 10 kW/cm.sup.2 would range between 15 and 20 kV/m.

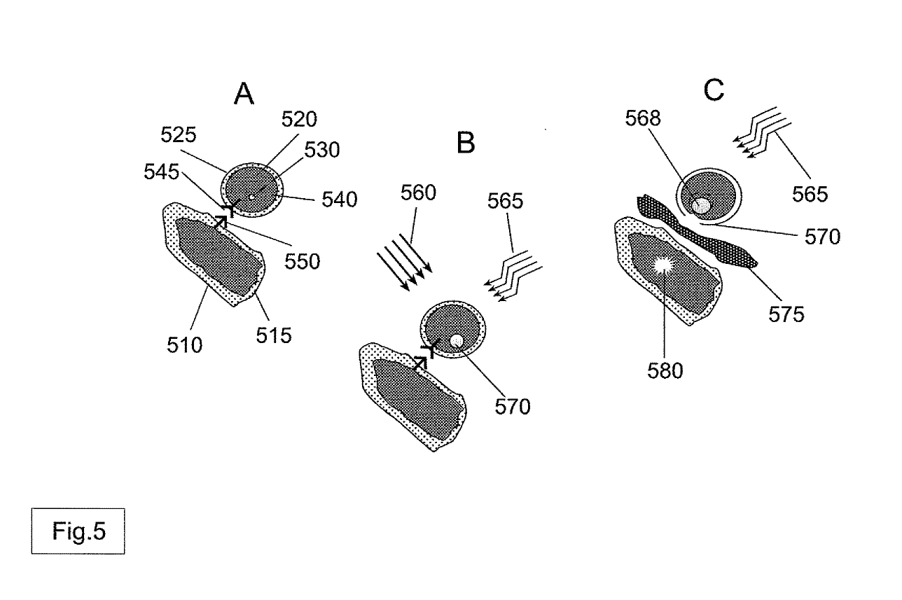

Rojas-Chapana et al., [7] exposed bacteria in a solution of 10 nm diameter carbon nanotubes to 10 second to an estimated power density of 500-W/cm.sup.2 2.45-GHz microwave irradiation (in-cavity power density). Following the exposure, the nanotubes penetrated into the bacteria lipid membrane and even into their liquid volume. The estimated microwave electric field was at most 4 kV/m. The drilled hole dimensions estimated from FIG. 5 of reference [10], is 10 nm diameter×20 nm thick, thus comprising about 10,000 organic bonds (assuming 20% organic material within the membrane). The authors claimed that the effect is related to field emission from the tips of the nanotubes.

The local electric field at the tip of rod-shaped nanoparticles (including CNTs) with a hemispherical tip can be estimated from the following equation [8]:

$${E}_{t} ( t ) = 1.2 ( 2.15 + \frac{2 l}{d} ) {E}_{0} ( t ) \qquad ( 2 )$$

Where E.sub.t and E.sub.0 are the time dependent tip and electromagnetic electric field, respectively, d and l are the nanoparticles diameter and length. According to the above equation, a multiwalled carbon nanotube whose diameter is 10 nm and its length is 500 nm would enhance the local microwave electric field by a factor of 150. The enhancement factor of a sphere is about 3.0 instead of about 5 according to equation (2).

The amount of CO₂ generated in the 20 cm.sup.3 test tube during the Kiel experiment [5] after five pulses is estimated as ˜10.sup.18 molecules. Assuming that the DALM molecules can enhance the local electric field by a factor of 100, exposing such molecules to ambient microwave radiation with power density of 1 MW/cm.sup.2 would induce an estimated peak electric fields reaching 1 MV/m in close proximity to these molecules. Similarly, the typical field enhancement factor attained at each multiwalled nanotube tips used is several hundreds [7] bringing the tip's local electric field to about 1 MV/m.

In both experiments there is a major discrepancy between the estimated electric field and the fields required, according to the literature, for inducing the observed phenomena by known mechanisms. For example, generating the amount of CO2 and the glow observed in Kiel experiment would require the induction of a glow discharge in the water, a phenomena which typically requires electric fields two orders of magnitude [9] (i.e., 100 MV/m) beyond the above estimates. Similarly, the threshold for field emission of electrons from the tips of the nanotubes requires ˜10.sup.8 V/m [10], two orders of magnitude beyond the above estimates.

Without wishing to be limited by a single hypothesis, it is predicted that the disintegration of NaHCO3 molecules described by Kiel [5] and the drilling in membrane described by Rojas-Chapana [7] result from the same phenomena, namely the production of ROS (reactive oxygen species) with absorbing nanoparticles and suitable microwave radiation. Without wishing to be bound to a specific theory, it is suggested that the enhanced microwave electric field (in the order of 1 MV/m) is somehow coupled to the water molecules and generates ROS. The generated ROS can react with the NaHCO3 molecule and generate CO2, and can break bonds in the bacteria membrane, eventually drilling a hole in and/or rupturing the membrane. Another plausible explanation (again without wishing to be limited by a single hypothesis) is that molecules at close proximity to the DALM molecules or to the nanotube are accelerated by the microwave electric field to very high speed. At such speed, the NaHCO3 molecules disintegrate upon collision with high speed water molecules, while the water and organic molecules break bonds in the bacteria membrane.

It is further predicted that electrons are not a major player in the observed phenomena. Even if a small fraction of electrons at the absorbing nanoparticles tip were accelerated to energies of 1 eV, their mean free path in water is a few nm, and thus cannot break bonds in organic molecules many nm away.

To support this prediction, the ROS production was validated and estimated using the spin trap technique [11]. Prior to microwave exposure, the nanotubes were mixed with water solution comprising 5-(diethoxyphosphoryl)-5-methyl-1-pyrroline-N-oxide (DEPMPO) from Calbiochem. This molecule interacts with various ROS types, yielding relatively stable (half-life of 17.7 min.) species such as DEPMPO-OOH with superoxide anion and DEPMPO-OH with hydroxyl radical. Each generated DEPMPO-x molecule preserves the interacting ROS spin and thus records (i.e., spin-traps) a fraction of the ROS amount generated by the nanotubes during the exposure. The DEPMPO-x spin spectrum is sampled using an Electron Paramagnetic Resonance (EPR) instrument.

During a typical experiment, about 200 microliter distilled DI water suspension comprising about 5*10¹⁰ carbon nanotubes (CNT), whose length to diameter ratio was about 20, and 100 microliter of DEPMPO were mixed in a 1 cm3 vial. The vial was exposed in a household microwave oven to 2.45 GHz microwave radiation (estimated incident power ˜30 W/cm2 or ˜1 kV/m) for 60 seconds period (dose=1.2 kJ/cm.sup.3).

Immediately after exposure, each vial was transferred to the EPR instrument and the spin spectrum was taken. The spin spectrum was also sampled for material from a vial with a CNT suspension before microwave exposure and also from a vial that was exposed to microwaves but which did not feature the CNT suspension. The generated ROS concentration was calculated by integrating the spectrum after subtracting the base level as described previously [11]. The measured ROS amount generated during 60 second exposure is equivalent to concentrations near 3*10.sup.-8 molar=1*10.sup.15 ROS molecules/cm.sup.3 or 100 ROS molecules per nanotubes (s.sub.0=1.6 ROS/sec). Equivalent results were obtained through microwave exposure of copper alloy particles whose size is estimated as 10 micron diameter and 50-100 micron long.

Without wishing to be bound by a specific theory, and based on a comparison of Kiel's observations and Rojas-Chapana's experiments, it is predicted that the ROS production is proportional to the microwave electric field to the fourth power (and intensity squared) and linear with the microwave dose.

The number of ROS generated at a tip of a nanotube is thus given by the following equation:

$$s = {{s}_{0} ( \frac{I}{{I}_{0}} )}^{2} {( \frac{β}{{β}_{0}} )}^{4} Δ t \qquad ( 3 )$$

Rojas-Chapana et al. exposed a nanotubes whose aspect ratio is β=50 to estimated power density of I=300 W/cm.sup.2 for Δt=10 seconds. In the above experiment the estimated power density is I.sub.0=30 W/cm.sup2 and the nanotubes aspect ratio was β.sub.0=20. According to equation 3 above, the nanotubes in the Rojas Chapana experiment generated 60,000 ROS molecules. It is predicted that such amount of generated excited species would be sufficient for breaking of 10,000 chemical bonds in the bacteria membrane, as estimated above to be sufficient for drilling a hole through the bacteria membrane, as observed by Rojas-Chapana.

The interaction of submerged conducive particles with microwaves has additional aspects as revealed by exposing submerged metal particles to microwave radiation. A cup comprising 200 ml cold (25 C) tap water, and about 10 mgr ˜20-50 micron long copper alloy rods at the cup bottom, was exposed to ˜30 W/cm.sup.3 2.45 GHz microwave radiation. After 20 second exposure, and one minute delay, a significant portion of the particles were observed floating on the surface of the water in large (5 mm) visible bright clusters. It is assumed that air comprising microbubbles attached to the particles caused them to float to the water's surface. Flotation of the particle would require an attached bubble whose volume is over ten times the particle volume (i.e., ˜10.sup.14 gas molecules). Thus, the microwave energized metal particles somehow extracted dissolved air molecules from estimated volume of 0.1 cubic mm of surrounding water, about 1000 times the particle volume. Comparative tests with DI water showed no flotation.

### Generating Nucleation Bubble

According to other preferred embodiments of the present invention, there is provided a non-thermal method for generating a nucleation bubble by exposing a one or more of nanoparticles in a liquid environment to an electromagnetic field. Without wishing to be limited by a single hypothesis, it is believed that the nanoparticles in the enhanced electromagnetic field induce the formation of ROS which in turn, generates a nucleation bubble. In certain aspects, the local electromagnetic field near the nanoparticle is at least 5 times the ambient electromagnetic field and is referred to herein as “enhanced”, such that preferably the nanoparticles are exposed to an enhanced electromagnetic field.

Without being bound to any specific theory, the term “non-thermal nucleation” means that the peak liquid immersed nanoparticle temperature during non-thermal generation of nucleation bubble does not exceed the boiling point of the liquid at the pressure within the nucleation site.

Preferably, the nanoparticles are first provided to an object to be treated, for example by being administered to and/or applied to the object, in which the object is in a liquid environment. The object is optionally part of a larger object, for example optionally and preferably comprising a portion of tissue to be treated in a body of a subject. The nanoparticles are then exposed to suitable microwave radiation. Preferably, a plurality of excited species is generated at close proximity to the nanoparticle(s), which is then released into the proximal liquid environment. These excited species preferably cause gas molecules to be generated in close proximity to the absorbing nanoparticle. These non-condensable gas molecules more preferably accumulate on the nanoparticles to form a nucleation bubble. In other aspects, the interaction of absorbing nanoparticles with the microwave radiation induces the accumulation of dissolved gas molecules on the nanoparticle.

In other aspects, liquid molecules are accelerated by the electromagnetic field in close proximity to the absorbing nanoparticle tip and in turn break bonds in dissolved and liquid molecules, thereby generating non-condensable gas molecules in close proximity to the absorbing nanoparticle.

Optionally and more preferably, these excited species then break weak bonds in molecules found in the liquid. The term “liquid” includes water, aqueous solution, non-aqueous solution, gel, semi-solid, suspension, dispersion, membrane, liquid comprising a solid matrix, etc. These molecules may be dissolved, suspended, emulsified, part of a gel matrix and so forth. This results in the generation of non-condensable gas molecules adjacent to the absorbing nanoparticle. The accumulation of non-condensable gas molecules on accumulation promoting site(s) on the absorbing nanoparticle surface results in the formation of a nucleation bubble. In certain aspects, the absorbing nanoparticles preferably comprise conductive material in the microwave frequencies such as metal, metal alloy, carbon, and certain semiconductors.

From the above described information, it is evident that exposing absorbing nanoparticles to suitable microwave radiation can generate at least several thousands non-condensable gas molecules. However, the generation of such amount of gas near an absorbing nanoparticle does not warrant the generation of a nucleation bubble. For example, the localized release of such amount of gas molecules which could form a 25 nm nanobubble will actually result in these gas molecules diffusing into the liquid continuum and dissolving in it.

The internal pressure inside a free floating nanobubble is called the Laplace pressure and is given by the following equation:

$${P}_{s} = \frac{2 {σ}_{st}}{{R}_{s}} + {P}_{∞} \qquad ( 3 )$$

Where Rₛ P_{∞} and σₛₜ are the nanobubble radius, ambient pressure and the liquid surface tension. For example, for a nucleation bubble whose radius is 50 nm, the internal pressure reaches 2.9 MPa.

Recently Attard et al. [12] have found that hydrophobic regions on surfaces tend to accumulate spontaneously and retain gas molecules as attached surface nanobubbles [12]. Thus, the gas molecules released from an absorbing nanoparticle near a hydrophobic site would attach to such a site and accumulate there. The nucleation bubble volume is expected to increase with the accumulating microwave energy density. They observed nanobubbles tendency to accumulate on hydrophobic surfaces and especially along micro-scratches or deviations in the surface. They further found that these surface nanobubbles are much less pressurized than would be expected according to the Laplace pressure. The surprising fact is that nucleation bubbles survive the Laplace pressure when attached to hydrophobic surfaces [12].

Agrawal et. al., [16] characterized the spontaneous accumulation of gas nanobubbles on hydrophobic patches. They found that nanobubbles accumulate spontaneously on submicron hydrophobic patches and that the thickness of the nanobubbles layer is a few tens of nanometers. Thus, the gas molecules released from an absorbing nanoparticle near a hydrophobic site would attach to such a site and accumulate there.

Farny et al [15] have validated experimentally that a free floating nanobubble may evolve into a microbubble (i.e. serve as nucleation bubble) if its radius is larger than the Blake radius. The Blake radius can be approximated from the relation [15]:

$${R}_{B} = \frac{0.77 {σ}_{s}}{{P}_{B} - {P}_{∞}} \qquad ( 4 )$$

Where P.sub B is the ultrasound rarefaction pressure needed to activate nucleation bubble.

Initially, it was thought that nanobubbles accumulated on hydrophobic surfaces which may serve as nucleation sites, especially since they are less pressurized, and thus expected to respond to lower ultrasound rarefaction pressure. However, recent experiments [17] demonstrated that nanobubbles whose size is equivalent to the Blake radius, on a flat, uniform and clean surface, are stable and do not serve as nucleation sites, even under strong (6 MPa) ultrasound insonation.

On the other hand, Bremond et. al., [14] exposed a silicon wafer section with etched 4 micron diameter hydrophobic wells and immersed in water to an ultrasound pulse that peaked at 1.4 MPa. He found that drying the wafer section with nitrogen before immersion “activated” the holes and led to repeatable cavitation and microbubble bubble forming following exposure. Without the activation procedure, ultrasound induced cavitation events were limited to a few repetitions.

Bremond et al. demonstrated microbubble generation at peak ultrasound overpressure of 1.4 MPa. This peak pressure corresponds with nanobubbles on the well inner surface, whose Blake radius would be larger than 40 nm according to the above relationship. The authors successfully simulated the microbubble dynamics using the Reileigh-Plesset equation, i.e. behavior of a normal microbubble.

It is predicted that Bremond's ability to generate microbubbles from surface nanobubbles stems from the internal surface structure of the wells they used, which comprised circumferential grooves. It is further predicted that nanobubble accumulation on sites selected from the group consisting of edges of hydrophobic sites, cracks, interface lines between materials, pits etc. may also serve as nucleation bubbles. In some embodiments, the present invention provides methods for generation of nanobubbles through exposure of absorbing nanoparticles to microwave radiation, thereby inducing intensive accumulation of non-condensable gas. It is further predicted that such intensive accumulation in suitable accumulation sites may increase the nanobubble size beyond the size of spontaneously forming nanobubbles, and thus enable generation of nucleation bubble and at lower ultrasound peak rarefaction pressures.

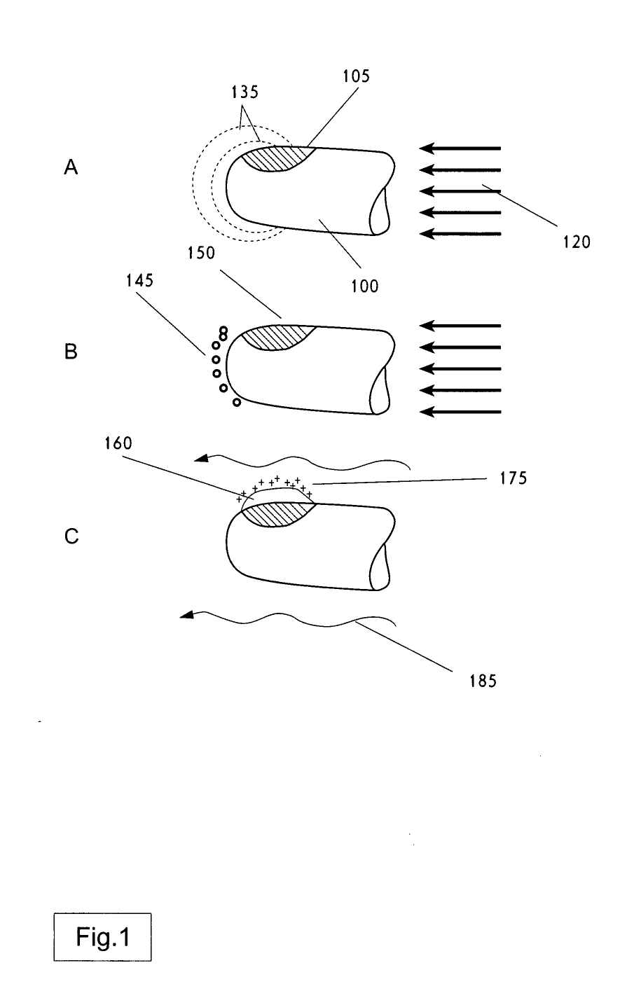

By way of illustration, one preferred method for generating a nucleation bubble is described in FIG. 1 one of the tips of a preferred but exemplary and illustrative absorbing nanoparticle with high aspect ratio 100 embodiment is schematically described in FIG. 1A. The absorbing nanoparticle 100, comprising at least one surface site 105 which promote the accumulation of gas molecules, is immersed in liquid environment and exposed to a period of microwave radiation 120. At the onset of microwave radiation 120, an electromagnetic field whose maximum level is more than 100 times higher than the ambient microwave electromagnetic field is preferably induced at close proximity to the nanoparticle 100 tip. The dashed lines 135 at close proximity to the absorbing nanoparticle 100 tip, represents iso-level fields line which are 100 and 50 times the strength of the ambient electric field in the liquid environment.

In reference to FIG. 1B, shortly after onset of the microwave radiation, excited molecular species 145 are optionally and preferably generated in the liquid at close proximity to the nanoparticle 100 tip. The excited species 145 interact with dissolved and suspended molecules including the liquid molecules, and in turn generate non-condensable gas molecules 150. A portion of the gas molecules 150 diffuse in close proximity to the nanoparticle 100 tip surface towards the site 105 and accumulate there.

In reference to FIG. 1C, at the end of the microwave radiation 120 period, the gas molecules have accumulated on the site 105 as at least one nanobubble 160. Beyond a certain size, the nanobubble 160 starts to interact with the ultrasound radiation 185, its size increasing slowly by the collection of dissolved gas molecules 175, and it becomes a nucleation bubble. If more than one nanobubble 160 exists on the site 105, they coalesce into a larger nanobubble 160 which may be larger than the absorbing nanoparticle 100 tip diameter.

In yet another aspect, the absorbing nanoparticle 100 may optionally and preferably be exposed to microwave radiation 120 short period before exposing to ultrasound energy 185 so as to increase the nanobubble 160 volume at the site and in turn reduce the ultrasound energy 185 peak pressure needed for inducing nucleation bubble.

PCT application WO 2006 051542 by the present inventor taught a method for efficient generation of thermal nucleation bubbles around electromagnetic radiation absorbing nanoparticles, which are exposed to electromagnetic radiation and in particular light. The present application by the present inventor provides an improvement over this method through the use of microwave radiation for generating the nucleation bubble. As in WO 2006 051542, the present invention may optionally also use ultrasound radiation in combination, for preventing the nucleation bubble from redissolving into the liquid and for causing it instead to grow through rectified diffusion. Rectified diffusion occurs when ultrasound energy causes supersaturated gas to be pumped into an existing small nanobubble, making the bubble increase in size.

### Nanoparticle Materials

According to preferred embodiments of the present invention, certain types of nanoparticles are preferred for producing nucleation bubbles through the application of suitable microwave radiation. According to preferred embodiments of the present invention nanoparticles are operable for enhanced absorption or enhanced scattering of microwave radiation as single nanoparticles or as clusters. Under certain embodiments, the nanoparticle interaction cross section at microwave radiation wavelength may optionally vary between 0.05 to 0.5 of their geometric cross section.

The size of the absorbing nanoparticles is preferably selected to be suitable for the method of use. For example, for treatment of tissue, such as in the body of a subject for example, the size of the nanoparticles is preferably selected to control their biological properties, more preferably including but not limited to a characteristic selected from the group consisting of the bio-distribution, penetration through vasculature and interstitial volume, and the blood clearance rate, or a combination thereof. The single nanoparticle size preferably is in the range of from about 10 to about 1000 nm. Suitable shapes of absorbing nanoparticles preferably include but are not limited to nanotubes, high aspect ratio rods or ellipsoids, etc. As a non-limiting example of a nanoparticle, a non-symmetrical nanoshell construction for absorbing nanoparticles optionally and preferably comprises a metal shell and silica core whose center does not coincide with the gold shell center [described by Wang et al. [11a]. The local electric field near such absorbing nanoparticles may enhance the ambient electric field by a factor of up to 60 for THz radiation.

In certain aspects, the absorbing nanoparticles may optionally comprise local nanometer sized structures which enhance the local electric field in their vicinity. For example, nanotubes, and especially multiwalled nanotubes comprise nanometer sized structures at their tips, which in turn, increase the field enhancement factor by a factor up to 2 from the value predicted for simple rod shaped absorbing nanoparticles.

Optionally and preferably, the absorbing nanoparticles comprise any metal, metal alloy, combinations of metals and non-metals, and non-metals. The nanoparticles may optionally comprise a single material, such as gold or carbon, or can be layered structures, such as silica shapes covered with gold shells. The layered nanoparticles include the asymmetric nanoshells configuration described by Wang et. al., [11a].

In some embodiments, the absorbing nanoparticles optionally include a metal core with a large aspect ratio, within a jacket made of a glass-like composition. In other embodiments the absorbing nanoparticles are optionally fabricated by microwire drawing technologies. In further aspects, the metal core diameter of the absorbing nanoparticles optionally ranges from about 10 nm to about 1000 nm.

According to preferred embodiments of the present invention, the absorbing nanoparticles have a nanotube shape. They are conductive, preferably multiwalled. Their diameter preferably varies from about 3 to about 20 nm and their longer dimension preferably varies from about 20 to about 2000 nm. The type of nanotubes is optionally and preferably selected from the group consisting of carbon nanotubes (CNT), Boron nitride nanotubes, BCN nanotubes, in which some carbon atoms were replaced by nitrogen and boron atoms (BCNT) silicone carbide nanotubes, bundles of single-wall carbon nanotubes, multi-wall carbon nanotubes, buckytubes, fullerene tubes, carbon fibrils, carbon nanotubules, carbon nanofibers, and combination thereof. The multi walled nanotubes preferably comprise multiple “walls” in their structural composition, according to the construction material. They may be in the shape of nanoscrolls, nanofibrils, nanovessels, nanocontainers, and combinations thereof. They may comprise a variety of lengths, diameters, chiralities, number of walls, and they may be either open or capped at their ends (for example see Bianco et al. in US patent application 20060199770).

In preferred embodiments of the present invention, the absorbing nanoparticle preferably includes at least one site comprising compositions and/or structures which promote the accumulation of gas molecules generated by its exposure to microwave radiation. In another aspect of the present invention, the site comprises a discontinuity suitable for accumulation of gas molecules as an attached nanobubble. In a preferred embodiment the surface of the absorbing nanoparticle preferably comprises at least one site suitable for accumulation of non-condensable gas molecules whose surface morphology selected from a group comprising: a crack, a depression, a linear edge, a pointed edge, a boundary between hydrophobic and hydrophilic materials, and a boundary between different surface characteristics.

According to preferred embodiments of the present invention nanoparticles are operable for enhanced absorption or enhanced scattering of microwave radiation as single nanoparticles or as clusters. Under certain embodiments, the nanoparticles interaction cross section at microwave radiation wavelength may vary between 0.05 to 0.5 of their geometric cross section.

### Biological Functionalization

According to some embodiments of the present invention, the nanoparticles are used for biological functions, for example for treatment of a subject or some cases for biological treatment outside of a subject (for example for treatment of biofilms as described in the next section).

According to some embodiments of the present invention, the nanoparticles are preferably adapted for use in vivo and/or in other biological applications. The size of the absorbing nanoparticles is crucial in controlling their biological properties, especially the bio-distribution, penetration through vasculature and interstitial volume, and the blood clearance rate. The single nanoparticle size could range from about 10 to about 1000 nm. For biological reasons, absorbing nanoparticles whose smaller dimension range between 10 and 100 nm will generally be favored over larger absorbing nanoparticles. An optimal absorbing nanoparticles size will be determined depending upon their shape, structure, materials, operation conditions and the details of the specific biological or industrial application.

For these embodiments, optionally and preferably one or more materials for biological functionalization are employed as described herein.

The present invention, in some embodiments, encompasses the use of absorbing nanoparticles comprising both coatings that are covalently bound to the surface of the absorbing nanoparticles and coatings that physically adhere to the surface of the absorbing particle. The latter method of binding will generally be more preferred. The coating material may comprise any of the elements. However, coatings comprising carbon, oxygen, nitrogen, hydrogen, sulfur and phosphorous are preferred.

In a preferred embodiment of the present invention, the absorbing nanoparticles are preferably coated with one or more materials which preferably provides at least one of the following functions: stabilize the absorbing nanoparticles in aqueous suspension to prevent their aggregation; prevent uptake of absorbing nanoparticles by the immune system if administered to a body of a subject; serve as an intermediate layer for attachment of targeting ligands; enhance nanoparticle transport through blood vessels and interstitial regions; maintain the capability of nanoparticles to effectively generate nucleation bubbles. In order to fulfill these functions, the coating may optionally contain one or more of a surfactant, linker, spacer, targeting ligand and encasing ligand, as will be explained below.

In a preferred embodiment, the absorbing nanoparticles are preferably coated with amphiphilic material such as one or more surfactants for example, so as to stabilize their suspension in aqueous solution and to prevent their spontaneous aggregation without rendering their ability to generate nucleation bubbles. Optionally, the surfactants may comprise one or more of polymeric or non-polymeric surfactants, preferably to stabilize the absorbing nanoparticles. Particularly desirable surfactants are block copolymers, especially block copolymers in which one block is poly(ethylene glycol) (PEG). Optionally the surfactants are proteins, modified proteins or other biological molecules as surfactants.

In another preferred embodiment, the coating preferably extends the circulation lifetime of the nanoparticles by minimizing uptake by the immune system when administered to a body of a subject. As a rule, nanoparticles are taken up by macrophages in the body or the RES (reticuloendothelial system), and are hence cleared by the immune system. The use of derivatives of poly(ethylene glycol) (PEG) to lengthen the circulation lifetime of particulate drug formulations is well known (Kumar, J. Pharm. Pharmaceut. Sci., 2000, 3, 234.). The attachment of PEG to gold nanoparticles is described for example by West et. al, in U.S. Pat. No. 6,530,944. Alternatively, the absorbing nanoparticles are coated with graft MPEG (methoxypolyethylene glycol), a hydrophilic polymer. MPEG coated liposomes demonstrated a circulation period of several days.

In yet another preferred aspect, the surfactant preferably serves as a platform for the attachment of other chemical species with desirable biological or chemical properties, such as targeting ligands. Thus, a coating material comprising reactive functional groups is desirable. Reactive functional groups suitable for the present invention include, but are not limited to hydroxyl groups, thiol groups, amine groups, hydroxyl, halo, cyano groups, carboxyl, and carbonyl groups, as well as carbohydrate groups etc.

Preferably the attached species such as targeting ligands and the coating comprise one or more reactive functional groups. The attachment process may be optionally performed using a linker (an endured connection) and a spacer (cleavable attachment). Both linker and spacer preferably have a pair of reactive functional groups.

The spacers may optionally comprise linker groups that are cleavable under the action of enzymes, acids, bases, and other chemical or biological entities. With such cleavable spacers, the absorbing nanoparticles may modify their properties over time. For example, before cleavage of the spacer, the nanoparticles may optionally have a strong affinity for certain cells, organs, or non-tissue material. After cleavage, they are preferably rapidly cleared from the body. Optionally and preferably, the nanoparticles comprise surfactants with modifiers attached directly or through linkers and spacers that are cleavable under the action of enzymes, bases, acids, or other chemical entities.

According to some embodiments, there are preferably provided nanoparticles to which a linker and optionally, a spacer, preferably equipped with reactive groups, is attached. In certain aspects, the linker or spacer species are attached to the nanoparticle coating.

According to some embodiments, the invention encompasses the use of absorbing nanoparticles to which one or more targeting ligands are attached, either through a chemical bond or through direct interaction with the nanoparticle surface. These targeting ligands are optionally operable to promote absorbing nanoparticles accumulation within the targeted biofilm. In other aspects, the targeting ligands are optionally operable for attachment onto the envelope of the targeted bacteria. For example, Zharov et. al., [19] used Protein A to attach gold nanoparticles to S. aureus bacteria.

The targeting ligands may optionally be attached directly to the surface of the absorbing nanoparticle or attached indirectly through the surfactant. The targeting ligands may optionally comprise any chemical group that binds to a “targeting receptor” associated with the targeted cells, tissue or non-tissue material. The targeting ligands can be derived from any synthetic, semi-synthetic, or naturally occurring chemical species. Materials or substances that can optionally serve as targeting ligands include but are not limited to amino acids, peptides, proteins, antibodies, antibody fragments, hormones, glycoproteins, lectins, sugars, saccharides, carbohydrates, vitamins, steroids, hormones, cofactors, and genetic material, including nucleosides, and nucleotides, and the like. The targeting ligand can optionally be either an independent molecule or a molecular fragment.

The receptors for which the targeting ligands have a special affinity are preferably chemical groups, proteins, or other species that are overexpressed and/or specifically expression by the targeted biofilm, or bacteria membrane. In general, terms the receptors can be any chemical feature of the targeted biofilm or bacteria. The receptors can also optionally be independent chemical entities in the blood or other body fluids, including externally administered drugs, drug components, or drug metabolites.

In a preferred embodiment of the present invention, the absorbing nanoparticles are preferably entrapped in liposome for enhanced extravasation, tissue penetration, and enhanced circulation lifetime. In another aspect, the liposome optionally and preferably comprises one or more targeting ligands. In other aspects, the absorbing nanoparticle maintains ability to generate nucleation bubble and its evolution, possibly into a microbubble, while in the liposome. In yet other aspects the encasing liposome smaller dimension ranges between 20 and 200 nm.

Clearly, slowing the short-term blood clearance of the absorbing nanoparticles has to be balanced against the preferable property that the nanoparticles are completely cleared from the body in the long term. For most types of the nanoparticles, their clearance from normal tissues such as the liver, kidney and brain within a reasonable period of time is essential to minimization of the nanoparticles long-term toxicity.

Solid nanoparticles suitable for the present invention are preferably robust and in turn are typically functionalized by attachment of amphiphilic material as described above to their surface, through chemical reactions. In contrast, the functionalization process for nanotubes is more complicated because of their delicate structure and inertness of their basal structure envelope. For example, certain functional molecules may be attached to the nanotube tips while their envelope may be left without coating. Bianco et al. in US patent 20060199770 describe various methods for CNT functionalization and targeting ligands attachment, mainly onto CNT tips.

### Microwave Induced Microbubble Generation

Unexpectedly, the present inventor has discovered that simultaneous exposure of an absorbing nanoparticle to suitable microwave radiation and ultrasound can generate a microbubble without generating a nuclei of vapor of liquid around the nanoparticle. Therefore, according to preferred embodiments of the present invention, there is also provided a method for preferably generating a microbubble in contact with an excited absorbing nanoparticle (an absorbing nanoparticle excited by an electromagnetic field) through the simultaneous application of ultrasound. Preferably, the parameters are selected such that the method comprises the generation of a microbubble without generating a mostly vapor nuclei around the nanoparticle and also such that the generation is non-thermal.

Preferably, the method further comprises first providing an absorbing nanoparticle to an object to be treated in a liquid environment, which may optionally comprise part of a larger object as previously described herein. The nanoparticle may optionally be provided through administration to the object, for example. The excitation of the nanoparticle preferably causes gas molecules to be generated. The accumulation of gas molecules on the absorbing nanoparticle preferably generates a nucleation bubble, which is more preferably increased in size through the application of ultrasound radiation.

Therefore, according to preferred embodiments of the present invention, there is also provided a method for optionally and preferably generating a microbubble in contact with an EM excited nanoparticle (a nanoparticle excited by an electromagnetic field) through the simultaneous application of ultrasound. Preferably, the parameters are selected such that the method comprises the generation of a microbubble without generating a vapor nuclei around the nanoparticle and also such that the generation is non-thermal.

Preferably, the method further comprises first providing a nanoparticle to an object to be treated in a liquid environment, which may optionally comprise part of a larger object as previously described herein. The nanoparticle may optionally be provided through administration to the object, for example. The excitation of the nanoparticle preferably causes gas molecules to be generated. The gas molecules preferably generate nucleation bubbles through accumulation on the nanoparticle, forming nucleation bubbles, which are more preferably increased in size through the application of ultrasound radiation.

Optionally and more preferably, the parameters for exposure of the nanoparticle to microwave radiation are such that reactive species are generated. The reactive species in turn preferably induce generation of non-condensable gases through interactions with the liquid environment. Accumulation of non-condensable gas molecules on sites on the absorbing nanoparticle preferably generates nucleation bubbles.

In a preferred embodiment, the present invention provides methods for generation of a microbubble optionally and preferably comprising: (a) administering multiple absorbing nanoparticle to the desired object in liquid environment so as to accumulate a cluster of the absorbing nanoparticles on the object; (b) exposing the cluster to microwave radiation with suitable parameters for generating excited species adjacent to each absorbing nanoparticle; (c) Generating nucleation bubbles through mechanisms comprising bond breaking processes adjacent to each absorbing nanoparticle of the cluster; (d) accumulation of non-condensable gas molecules on accumulation promoting site(s) on the absorbing nanoparticle surface until they constitute a nucleation bubble; (e) increasing the nucleation bubble size through interaction with ultrasound thereby evolving it into a microbubble.

The nucleation bubbles that would be generated following exposure of absorbing nanoparticles to microwave radiation comprise relatively cool gases and thus expand slowly or not at all. On the other hand, as found by Attard [12], nanobubbles located at a distance of less than about 100-200 nm attract to each other and eventually coalesce. Thus, exposing a cluster of standing nanobubbles to ultrasound will expand the nanobubbles and narrow the gap between adjacent nanobubbles below the attraction threshold.

In preferred embodiments of the present invention, a cluster of nanobubbles is optionally and preferably generated, each nanobubble being adjacent to a respective absorbing nanoparticle in a cluster. Coalescence of the nanobubbles into at least one larger nucleation bubble occurs as a result of ultrasound induced expansion, through rectified diffusion, and mutual attraction. Rectified diffusion occurs when ultrasound energy causes supersaturated gas to be pumped into an existing small nanobubble, making the bubble increase in size. This process in turn reduces the threshold for microbubble generation from absorbing nanoparticles exposed simultaneously to microwave radiation and ultrasound.

In preferred embodiments of the present invention the size of the nucleation bubble is increased through interaction with ultrasound radiation. The method then optionally and preferably further comprises (e) coalescence of the nanobubbles into at least one larger nucleation bubble as a result of ultrasound induced expansion and mutual attraction; (f) increasing the nucleation bubble size into a microbubble through rectified diffusion.

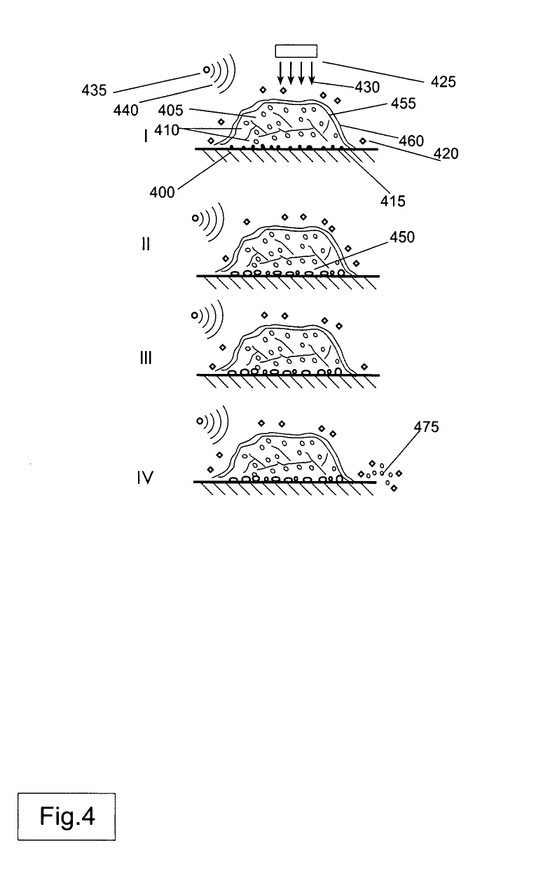

Krasovitsky et al. [18] conducted 1-D numerical simulations have indicated that exposing ten expanding vapor nucleation bubbles (initial laser energy absorbed in nanoparticles ˜1*10.sup.-12 J) to 1.1 MHz ultrasound radiation whose peak ultrasound overpressure much less than 0.2 MPa is sufficient for evolving the nanobubbles cluster into a united stable microbubble. FIG. 9 in the article shows the equivalent radius evolution of 10 clustered nucleation bubbles exposed to 0.2 MPa. FIG. 4 shows the evolution of a discrete nanobubble, exposed to 0.3 MPa and similar conditions. After coalescing (onset time ˜100 nsec), the united coalesced bubble steadily evolves into microbubble by rectified diffusion. On the contrary, a discrete expanding nucleation bubble exposed to the same conditions quickly decays and disappears. Their work provides the basis for the derivation of the ultrasound operating parameters of the present invention in some embodiments as described herein.

### Operating Parameters for Microbubble Generation

The operating parameters of the ultrasound intensity and peak pressure specified in the present invention are derived from the simulation results in [18]. The operating parameters of the microwave intensity are based on the experiments described in [5,6], the predicted reduction in microwave intensity due to combined ultrasound effect and experiments conducted by the present inventor.

According to preferred embodiments of the present invention, the microwave frequency is between 20 MHz and 1000 GHz, and more preferably between 100 MHz and 3 GHz. According to a preferred embodiment of the present invention, the electromagnetic source pulse width may optionally and preferably vary between 10 nanosecond and 30 milliseconds and more preferably between 0.01 and 10 microsecond. The average microwave power density optionally and preferably ranges between 0.1 kW/cm2 to 1 MW/cm2, and more preferably between 1 kW/cm2 and 100 kW/cm2.

In preferred embodiment the microwave radiation may optionally and preferably be employed in a mode selected from a group of single pulse mode, pulse train mode, repeated sequence mode, or any other time sequence suitable for inducing nucleation bubbles growth around absorbing nanoparticles which are exposed simultaneously to appropriate ultrasound radiation.

Preferably, the ultrasound source frequency varies between about 20 kHz and 10 MHz and more preferably between 0.5 and about 10 MHz. In general, frequency for therapeutic ultrasound preferably ranges between about 0.75 and about 3 MHz, with from about 1 and about 2 MHz being more preferred. In addition, energy levels may vary from about 0.5 Watt (W) per square centimeter (cm.sup.2) to about 20 W/cm.sup.2, with energy levels of from about 0.5 to about 2.5 W/cm.sup.2 being preferred.

As mentioned above, a phase delay of the ultrasound peak rarefaction of ⅛ of a cycle (45 deg.) in respect to the laser pulse onset, has been found by Farny as optimal for generating nanobubbles. In other aspects, the ultrasound radiation may be synchronized with the microwave radiation at a specific location within the region where the generation of nucleation bubbles is desired. For example, the ultrasound peak rarefaction pressure may be at a phase delay of ⅛ of a cycle in respect to the peak microwave electric field. In other aspects, the ultrasound peak rarefaction delay may optionally and preferably be varied such that various locations within the region are exposed to a phase delay of about ⅛ of a cycle.

### Nucleation Bubbles Generation In Vivo

As mentioned above, inducing nucleation bubbles using microwave electromagnetic radiation have significant advantages over photonic radiation due to its deep penetration into typical tissue (up to 10 cm and typically 2-5 cm for 1 GHz radiation). Microwave radiation, and especially in the low GHz, can penetrate through the body. The present invention optionally and preferably provides methods for a group of non-invasive treatments which employ controlled generation of nucleation bubbles.

In certain embodiments, the ultrasound energy may optionally and preferably be introduced to the targeted tissue (generally, superficial tissue) region by positioning external ultrasound source. However, deep tissue treatment requires focusing the ultrasonic energy so that it is preferentially directed within a focal zone. Examples for such sources are HIFU sources composed of an array of ultrasound sub sources. Alternatively, the ultrasonic energy may optionally be applied via interstitial probes, intravascular ultrasound catheters, or endoluminal catheters, typically composed of mechanically insulated metal wire.

Under certain aspects, the microwave source is optionally and preferably so arranged so as to efficiently couple the microwave energy into the region of interest. On other aspects the coupling is optionally and preferably attained by an array of microwave sources such as those described by Xiang et al in US Patent application 20070168001. In other aspects, the electromagnetic radiation source is optionally and preferably coupled to the specific patient region by a waveguide equipped with a matching terminating emitter, a metal wire structure, or a compact RF applicator, such as the device described by Kopti et al [8] coupled to a catheter.

In certain aspects, the nucleation bubbles optionally and preferably comprise non-condensable gas molecules generated through the dissociation of naturally occurring organic molecules dissolved in the fluids comprised within the region of patient. In other aspects the gas molecules are optionally dissolved gases such as oxygen and nitrogen which are collected from the liquid phase and accumulate on the suitable site on the absorbing nanoparticles.

### REFERENCES

[1] V. R. Stewart, P. S. Sidhu. New directions in ultrasound: microbubble contrast. Br J Radiol. 79 (2006) 188-94.
[2] A. L Kalibanov, Microbubble contrast agents: targeted ultrasound imaging and ultrasound-assisted drug-delivery applications. Invest Radiol. v 41 n 3 p. 354-362 (2006).
[3] N. Rapoport, “Ultrasound assisted drug release from Duxorubicin filled nanobubbles,” J. National Cancer Institute (Oct. 7, 2007).
[4] R. V. Sabariego, L. Landesa et. al., “Design of a microwave array hyperthermia applicator with a semicircular reflector V 37 n 1 p. 612-617 (1999).
[5] J. L. Kiel, R. L. Shernam, et. Al., “Pulsed microwave induced light, sound and electrical discharge enhanced by a biopolymer,” Bioelectromagnetics, v 20 n 4 p. 216 223 (1999).
[6] J. L. Kiel, R. E. Sutter, et al., “Directed Killing of Anthrax Spores by Microwave-Induced Cavitation,” IEEE Trns. Plasma Sci. V. 30, n. 4, p. 1487-1496 (2002).
[6a] L. Perreux and A. Loupy “Tentative rationalization of microwave effects in organic synthesis according to the reaction medium and mechanistic considerations,” Tetrahedron Report 588, Tetrahedron v 57 p. 9199-9223 (2001).
[7] J. A. Rojas-Chapana et. al., “Enhanced Introduction of Gold Nanoparticles into Vital Acidothiobacillus ferrooxidans by Carbon Nanotube-based Microwave Electroporation,” NanoLetters v 3 n 5 p. 985-988 (2004).
[8] N. de Jonge and J-M Bonard, “Carbon nanotube electron sources and applications,” Phil. Trans. R. Soc. Lond. A V 362 p. 2239-2266 (2004)
[9] Katsuki, S. Leipold, F “Plasma formation and electrical breakdown in water Plasma Science, 2002. ICOPS 2002. IEEE Conference p. 221-222
[10] J-M Bonard, M. Croci, et. al., “Carbon nanotube films as electron field emitters,” Carbon V 40 p. 1715-1728 (2002).
[11] Murrant, C. L., and Reid, M. B. Microsc. Res. Tech. V 55, p. 236-248 (2001).
[11a] [7] H. Wang, Y. Wu et. al., “Symmetry breaking in individual plasmonic nanoparticles,” PNAS, p. 10856-10860, Jul. 18, 2006.
[12] P. Attard, M. P. Moody et al., “Nanobubbles: the big picture,” Physica A v 314 p. 696-705 (2002).
[13] C. H. Farny, T. Wu, et al., “Nuclating Cavitation from Laser-Illuminated Nano-particles,” Acoustic Research on line v 24 20.6 2005.
[14] N. Bremond, M. Arora, et. al. “Controlled Multibubble Surface Cavitation,” Phys. Rev. Lett. V 96, p. 224501 (June 2006)
[15] T. Wu, Bubble mediated focused ultrasound nucleation, cavitation dynamics and lesion prediction, PhD thesis, Boston University, College of Engineering, 2007.
[16] A. Agrawal, J. Park, et. al., “Controlling the Location and Spatial Extent of Nanobubbles Using Hydrophobically Nanopatterned Surfaces,” MIT Report HML 05-P-09 (2005).
[17] Nanobubbles on smooth surface do not serve as nucleation sites.
[18] B. Krasovitsky, H. Kislev and E. Kimmel “PHOTOTHERMAL AND ACOUSTICAL INDUCED MICROBUBBLE GENERATION AND GROWTH,” Ultrasonics (2007) To be published.
[19] V. P. Zharov, R. R. Letfullin, et al., “Microbubbles-overlapping mode for laser killing of cancer cells with absorbing nanoparticles clusters,” J. Phys. D: Appl. Phys. v 38 pp. 2571-2581 (2005).

### Section 2—Applications of Microbubbles for Treatment of Biofilms

This Section relates to the treatment of biofilms with the methods according to the present invention. A first example relates to treatment of a biofilm in a tissue, such as for example in the body of a subject. A second example relates to treatment of biofilms on various surfaces which are not part of living tissue, such as for example for industrial applications.

In a public announcement by the U.S. National Institutes of Health, it was stated that over 80% of microbial infections in the human body are mediated by biofilms [a1]. Incredibly, though the vast majority of the world's microorganisms are thought to be surface associated, the prevalence and significance of microbial biofilms have only recently captured the attention of the scientific community.

Biofilm protected bacteria are responsible for a legion of common ailments, such as lung infection in patients with cystic fibrosis (CF), otitis media (commonly known as ear infection), periodontitis, wound infection in chronic wounds, burn patients and diabetic tissue (particularly foot tissue), infections caused by a variety of surgical implants, endocarditis, urinary tract infections, and many others.

A recent study [a2] has indicated that more than 19 million chronic wound events occur globally per year. Most of these wounds are contaminated by biofilm forming bacteria [a3]. The natural progression of wound healing is delayed by biofilms.

Another recent study [a4] has indicated that more than a million antibiotics resistant hospitalization induced events have occurred in the US alone in 2006. Zumeris in US Patent Application 20050268921 indicates a strong relationship between bacterial colonies capable of developing biofilm and their ability to develop antibiotics resistance.

Bacteria may exist within a fluid media as individual cells or may form on a surface bounding the fluid medium in a conglomerate of microbial organisms termed a biofilm. The biofilm structure can provide a competitive advantage for the microorganisms since they can reproduce, are accessible to a wider variety of nutrients and oxygen conditions, are not washed away, and are less sensitive to antimicrobial agents. Therefore, the bacteria live at a lower metabolic state in the biofilm than when in planktonic form. The formation of the biofilm is also accompanied by the production of exo-polymeric materials (polysaccharides, polyuronic acids, alginates, glycoproteins, and proteins) which together with the cells form thick layers of differentiated structures separated by water-filled spaces. The resident microorganisms may be individual species of microbial cells or mixed communities of microbial cells, which may include aerobic and anaerobic bacteria, algae, protozoa, and fungi.

A model of stagewise bacterial biofilm development general to many motile bacterial species is suggested by Stoodley et al. Annu. Rev. Microbiol. 2002 pp. 187-209. Stage I involves reversible attachment to a surface. In maturing to stage II, the bacterial cells secrete exopolymeric substances and attachment becomes irreversible. Stage III denotes early maturity, as three-dimensional architecture begins to appear within the biofilm. Complex architecture is found as growth continues to stage IV, generally considered as fully mature. Stage V is called the “dispersion” stage, where structures within the biofilm develop hollow cavities filled with hypermotile cells that are released upon opening of the channels to spread and begin the process anew.

Bacteria in the biofilm form strong chemical bonds with surface carbohydrate moieties. The exopolymers encase the bacteria in a manner that leaves tunnels or channels through which the overlying fluid medium can circulate. In this way, the bacteria are protected from the dangers of the fluid medium, can receive nutrients, and rid themselves of waste.

When the biofilm is formed on living tissue, the biochemical products and toxic wastes it secretes may affect the tissue surface to produce an inflammatory state and areas of chronic infection, such as chronic ear disease, osteomyelitis, chronic tonsillitis, prostatitis, vaginitis, and calculi, as in the kidney. In many cases, chronic sinusitis appears to be an inflammatory disease of the lining mucosal, rather than the disease of bacteria-invading tissue.

The biofilm insulates the embedded bacteria from biocides contained in the proximal fluid layer so that normal concentrations of antibiotics or the like, which would kill the bacteria if they were in a planktonic state, have little or no effect on the bacteria of a biofilm. For example, sodium hypochlorite, an oxidizing biocide of unparalleled efficacy, requires a 600-fold concentration increase to kill biofilms of wild-type Pseudomonas aeruginosa compared to equivalent bacteria lacking ability to generate biofilm. Ampicillin has a 2 μg/mL minimum inhibitory concentration (MIC) against a β-lactamase negative strain of Klebsiella pneumoniae. The same strain, when grown as a biofilm, survives well when treated for 4 hours with 5000 μg/mL ampicillin, a 2500-fold increase in MIC.

There are three hypotheses that explain the antibiotic resistance of bacteria when contained in biofilms. The first hypothesis, termed penetration limitation, suggests that only the surface layers of a biofilm are exposed to a lethal dose of the antibiotic due to a reaction-diffusion barrier that limits transport of the antibiotic into the biofilm. A second hypothesis states that varied microenvironments within parts of the biofilm allow for several layers of defense against antimicrobial agents. Biofilms are highly structured, often possessing water channels for shuttling nutrients and waste products to and from the interior portions of the biofilm. In addition, oxygen is often consumed at the surface of such biofilms, providing for anaerobic pockets which can antagonize the action of some antimicrobials. A third hypothesis is that differential gene expression in biofilm bacteria aids somehow in bacterial survival. For example, P. aeruginosa biofilms express more than 800 proteins at a difference at least six-fold from planktonic levels at some point in their maturation. Such pronounced physiological changes certainly contribute to the relative inefficacy of antimicrobials which are used to treat planktonic bacteria.

Various approaches have been suggested to fight biofilm on accessible body cavities and medical implant surfaces during its development: (I) preventing the surface attachment of a bacteria conglomerate; (II) Preventing conglomerate transition into a biofilm; (III) preventing signaling between bacteria within the biofilm (quorum sensing), a key biofilm protection mechanism; and (IV) Inducing minor or major tearing of the biofilm envelope through mechanical or chemical methods.

Past efforts to disrupt the biofilm by disrupting it have included treatment with chemical compounds such as antibiotics, chemical agents directed at dissolving or breaking up the polysaccharide binders such as surfactants, enzymes, denaturing agents, and the like. However, biofilms display tremendous resistance to traditional antimicrobial therapies and surprising fluidity in regulating expression of their genes in concert with one another to efficiently deal with rapidly changing and actively hostile environments.

Since the early experiments reported by Mayo Clinic in 1997, it is well known that ultrasound activated microbubbles are effective against biofilms. Initially, it was considered that the biofilm is disrupted by cavitation. Ultrasound alone has many drawbacks for treatment of biofilms, however, as the application of extracorporeal ultrasound has been demonstrated as insufficient to prevent biofilm growth and/or to reduce the efficacy of its protection [a13].

### EXAMPLE 1

### Treatment of a Biofilm in a Tissue

This Example relates to various optional, exemplary embodiments of methods according to the present invention for treating a biofilm when present in a tissue, such as for example in a body of a subject.

Previous attempts have been made to apply ultrasound with microbubbles for removing or disrupting biofilms. Unfortunately, these attempts all have significant drawbacks. For example, Rontal in US Patent Application 20060069343 suggested removing biofilm from accessible tissue surfaces by introducing encapsulated microbubbles, such as contrast agents, to the surfaces. The combined action introduces disruptive materials to the biofilm while the ultrasound generating cavitations (i.e., microjets) near microbubbles which tend to tear off portions of the biofilm. Contrast agents may also be introduced to the circulation system and may be equipped with targeting ligands for attachment to biofilms at inaccessible patient regions. Unfortunately, contrast agents cannot access biofilm structures within a diseased tissue. Also, contrast agents are a high-cost product with very short lifetime in body fluids.

Brewer in US Patent application 20070011836 suggested exposing microbubbles introduced into the gum area to ultrasound in order to reduce the biofilm buildup on the tooth and gum surfaces. He suggested introducing the microbubbles by adding them to a dental fluid, or to generate them by interactions of the brush fibers with the dental surfaces. Brewer claims that shear forces sufficient to reduce the biofilm buildup may be in the order of several tens Pa. This estimate is supported by measurement incorporated by Labib et al. in US Patent application 20050126599 who estimated the shear forces needed for disrupting membrane fouling biofilm, by 50 Pa. Such shear forces may be generated by pulsating the microbubbles without rupturing them using ultrasound operating at suitable mechanical index. Brewer suggested administering the ultrasound energy to the tooth region by an ultrasound guide. He claims that the ultrasound may induce forced flow of fluid through acoustic streaming effect. He further claims that the generated microbubbles generated by the brush fibers may migrate several millimeters before disappearing. Unless the blood vessel is highly perforated, microbubbles cannot penetrate through blood vessels and obviously not through the interstitium.

Recently, drug carrying nanocapsules [a6] have been suggested as method for fighting cancer after rupture by therapeutic ultrasound. Nanocapsules with a size of several hundred nanometers have better migration properties compared to contrast agent microbubbles. In principle such nanocapsules with suitable ligands may attach to biofilm structure could serve as nucleation sites for microbubbles production. However, their size (typically 100-200 nm) limits their migration rate through blood vessels and especially through the intercellular space. Also generation of microbubbles from discrete encapsulated nanobubbles requires high ultrasound rarefaction pressure.

In contrast to the above, absorbing nanoparticles of the present invention can penetrate through blood vessels and the interstitium and attach to biofilms within the diseased tissue. For example, nanoparticles whose size is below 100 nm may easily penetrate through micron sized holes in the tissue and blood vessels generated by toxin released from S. aureus within the diseased tissue [a7].

It was unexpectedly found that administering absorbing nanoparticles to biofilms and generating microbubbles adjacent to the biofilm can be used to disrupt the biofilm protection for the host bacteria. Further, it was found that by damaging the biofilm structure, tens of bacteria may be killed per a nanoparticle by permitting an antibiotic to enter through the biofilm.

According to some embodiments the present invention provides absorbing nanoparticles for antimicrobial treatment of a body tissue contaminated with bacterial biofilms, by imparting energy to the biofilm through ultrasound interaction with microbubbles generated in the area of and preferably adjacent to the biofilm. Without wishing to be limited by a single hypothesis, the biofilm structures may be affected through several mechanisms which include but are not limited to damaging the envelope layer of the biofilm, damaging the internal biofilm structure, agitating the biofilm content and thus increasing the bacterial metabolism rate, and applying shear forces on the biofilm structure, thus reducing or disrupting its adhesion to the host surface. It is anticipated (again without wishing to be limited by a single hypothesis) that the dominant rendering mechanism varies with the time-dependent distribution of the absorbing nanoparticles on the biofilm surface or within the biofilm volume.

When a contrast agent microbubble (such as Albunex™ microbubbles), whose size is 2-5 micron, is exposed to ultrasound radiation, it pulsates at the ultrasound frequency while inducing microstreaming around it [8]. For example, a 3 micron microbubble exposed to ultrasound power of 3 W/cm2 would generate at a distance of 0.5 micron, local flow velocity is Order of (1 cm/sec) and at the same time inducing local shear stresses of Order of (1000 Pa). Marmoutant et al. [a9] have found that exposing a cell substitute or mimicking particle to a pulsating microbubble temporarily causes the membrane to become leaky (i.e. having disrupted membrane continuity). As mentioned above, a biofilm is dramatically more sensitive to mechanical vibration and becomes disrupted at 50 Pa, which would enable antibiotics access in close proximity to the pulsating microbubble contact point.

Several authors claimed that the biofilm has a cellular structure and thus its breaching it at a single point is not sufficient to introduce biocides into the hosted bacteria [a10]. Thus, it is important that each biofilm structure would suffer a multi-point attack so as to ensure antibiotic access to the host bacteria.

It was unexpectedly discovered that administering absorbing nanoparticles of the present invention, preferably with ligands to a biofilm provides a unique attack mechanism which is not suggested or taught by the background art.

As a non-limiting, exemplary description, the application of a method according to the present invention for the disruption of a biofilm is described with regard to an illustrative bacterial species, S. aureus. In recoverable ulcer tissue, the volumetric concentration of S. aureus in diabetic foot tissue does not exceed 5·10⁷ bacterial/cm³ in a living tissue. Beyond this volumetric level, the tissue dies [a11]. In certain isolates, taken from diabetic foot ulcers, the S. aureus bacteria build biofilms within diabetic foot ulcer whose typical diameter is ˜15μ and length of 50μ. At such bacterial concentrations, the sum of lengths of biofilm structures within one cubic cm would not exceed 10⁶ microns.

The ultrasound absorption rate of microbubbles is proportional to their concentration. To avoid ultrasound screening by the generated microbubbles, the microbubbles volumetric density preferably does not exceed 3·10⁵ microbubbles/cm³ which is roughly equivalent to attenuation of 50%/cm at 1 MHz [a12]. Using the above data, the averaged linear microbubbles density on or within a biofilm would be 1 microbubble per 3 microns. The number of breaches that would be induced on the biofilm envelope at such linear density is therefore sufficient to defeat the cellular biofilm structure.

Thus, according to some embodiments, the present invention optionally and preferably provides a method to treat tissue volume contaminated with bacterial biofilm by using ultrasound and microbubbles, without self masking the ultrasound energy by the pulsating microbubbles. The generated microbubbles are preferably localized at close proximity to the biofilm surface and act specifically on the biofilm, defeating its protection while minimizing collateral damage within the tissue volume. Previous methods are not specific to the biofilm itself and thus are suitable only to remove biofilm from accessible contaminated tissue surfaces, as otherwise damage to the tissue itself could result.

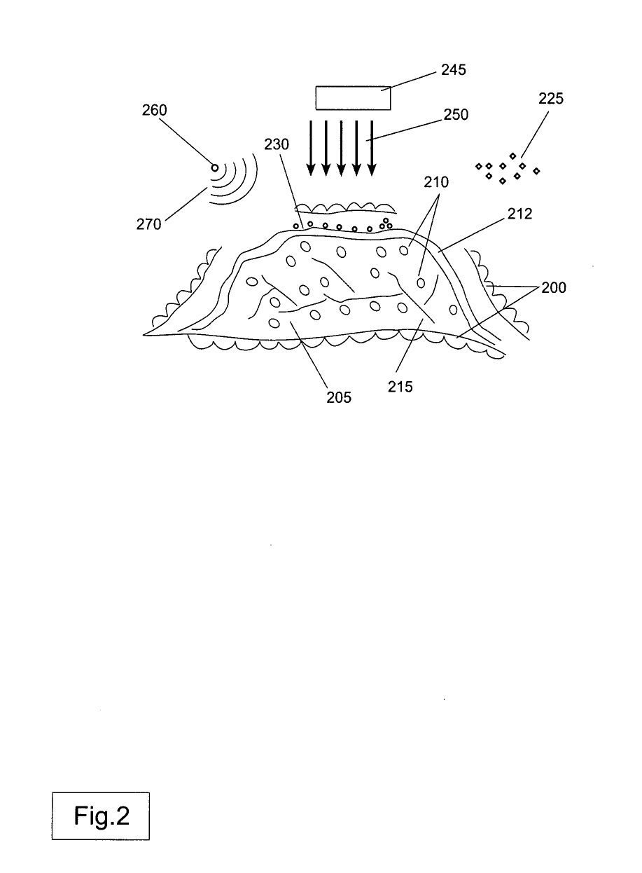

By way of illustration, FIG. 2 depicts one optional, exemplary but preferred embodiment of a treatment against a biofilm hosted bacteria. A biofilm structure 205 with polysaccharide envelope 212 has developed in the interstitium between cells membranes 200 of a bacterial contaminated tissue. The biofilm hosts a bacterial colony 210 located within a cellular structure comprising membranes 215. The contaminated tissue 200 is administered with a mixture of suitable antibiotics 225. The contaminated tissue 200 is also preferably administered suitable absorbing nanoparticles 230, which are preferably fabricated to increase their tendency to attach to biofilm envelope 212, preferably as clusters. An electromagnetic source 245 is preferably operable to expose the biofilm structure 205 a suitable period of electromagnetic radiation 250. An ultrasound source 260 is operable to exposure the biofilm structure 205 to suitable ultrasound energy 270 for the treatment.

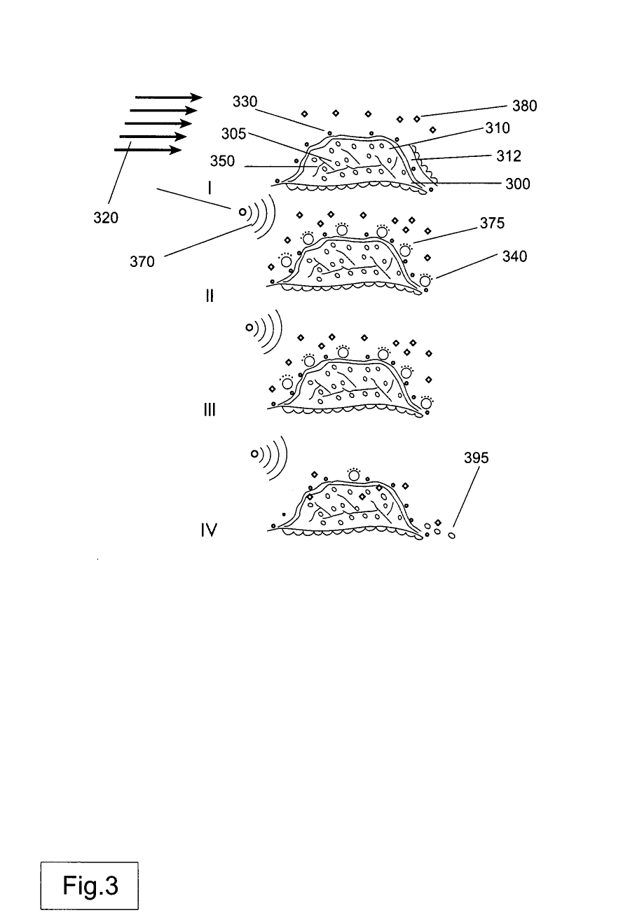

A detailed view of a biofilm section during a preferred treatment is illustrated in FIG. 3. The biofilm 305 hosts bacterial colony 310 protected within a polysaccharide envelope 312 and adhered to a contaminated tissue 300. In stage I, a portion of the absorbing nanoparticles 330 preferably accumulate in the biofilm envelope 312, preferably as clusters.

Each time the nanoparticles 330 on the biofilm 305 are exposed to electromagnetic radiation 320, a nucleation bubble is generated around the nanoparticles 330. Each nucleation bubble may evolve into microbubbles 340 through interaction with the ultrasound radiation 370, through rectified diffusion. Microbubbles 340 also pulsate under due to ultrasound radiation 370 (stage II). The pulsating microbubbles 340 induce microstreaming in close proximity to the biofilm envelope 312, thereby inducing damage to the biofilm envelope 312 (stage III).

Continued exposure to ultrasound energy 370 increases the damage to the biofilm envelope 312, possibly agitating the biofilm 305 content and also possibly damaging the biofilm internal membranes 350. Furthermore, the microbubbles 340 may damage the adhesion of biofilm 305 to the diseased tissue 300 surface, enabling access of antibiotics 380 to its host bacterial colony 310. Upon contact, antibiotics 380 kills at least a portion of the bacterial colony 310 and later eradicate at least a portion of the released planktonic bacteria 395 (stage IV).

In certain aspects, the nanoparticles in the cluster 14 attach to the biofilm 16 surface using ligands specific to the biofilm. In other aspects the nanoparticles may accumulate at close proximity to the biofilm structure through mechanism involving flow properties of the tissue contaminated with the biofilm. For example absorbing nanoparticles 14 may brought by external force acting on their suspension to flow through perforations induced in diseased tissue 1 by toxin released from the contaminating S. aureus biofilms.

Preferably, the dominating ultrasound source frequency varies between about 0.1 and about 10 MHz. The dominant frequency may be fixed or scan a range of frequencies continually to ensure optimum effect on the biofilms within the given treatment scenario. In general, frequency for therapeutic ultrasound preferably ranges between about 0.75 and about 3 MHz, with from about 1 and about 2 MHz being more preferred for deep treatments. In addition, energy levels may vary from about 0.5 Watt (W) per square centimeter (cm.sup.2) to about 5.0 W/cm.sup.2, with energy levels of from about 0.5 to about 2.5 W/cm.sup.2 being preferred. In terms of mechanical index, the MI optimal for fighting biofilm may range between 0.1 and 1.5 and more preferably between 0.3 and 0.8.

Furthermore, without wishing to be limited by a single hypothesis, it is predicted that after inducing multiple damage points in the biofilm structure, it may disperse naturally, such that even if a method according to the present invention does not directly disrupt the biofilm, it may optionally do so indirectly. Hazan [a13] has found that vibrating solid surfaces at nanometric amplitude is sufficient to avoid biofilm growth on these surfaces and their vicinity. Kane Biotech Ltd. Group observed spontaneous dispersion [a14] of the biofilm following limited damage induced by the Dispersin B enzyme. In addition they observed effective attack of bacteriophages on the biofilm following treatment with this enzyme.

In certain aspects, the shear forces induced by the microbubbles generated by methods provided by the present invention induce a certain level of damage to the adjacent biofilm structures which does not provide sufficient access of antibiotics for eradicating the host bacterial colony. However the damage to the biofilm triggers certain bacterial colony mechanisms (such as quorum sensing) which cause the biofilm to disperse (stop forming) after sensing the mechanical damage.

Henzer and Giskov in J. Clin Invest 112: 1300 (2003) administered several group of materials, (quorum sensing molecules) to biofilm forming P. aeroginosa bacteria. They grew biofilm on surfaces in a flow cell using the treated bacteria and untreated bacteria. After the biofilm matured, they administered tobramicyn (antibiotics effective against P. aeroginosa) to both types of biofilm samples. Using confocal microscopy they observed complete bacteria death only within the treated antibiotics sample. Apparently, the quorum sensing molecules were attached to these bacteria which in turn “joined” the biofilm colony.

Nanoparticles (or clusters thereof) are preferably attached or at least optionally brought into proximity to biofilm forming bacteria. EM radiation, preferably microwaves, and ultrasound may optionally be applied then to at least disrupt the process of biofilm construction. However, if the biofilm is actually constructed, the exposure of host bacteria, having nanoparticles attached to them and/or in their immediate environment within the biofilm, to EM radiation and ultrasound causes a microbubble to be generated. These microbubbles pulsate under ultrasound radiation applied to the biofilm surface, generating shear stresses within the biofilm structure and thereby enabling access of antibiotics to the hosted bacteria, leading to at least to their partial kill

According to some embodiments, the present invention provides methods for eradicating at least one biofilm hosted bacterial colonies comprising (a) administering absorbing nanoparticles operable for attachment onto biofilm forming bacteria; (b) waiting a suitable period of time until sufficient number of bacteria carrying absorbing nanoparticles accumulate within the biofilm; (c) exposing the biofilm to simultaneous electromagnetic radiation and ultrasound radiation thereby generating at least one microbubble around one or more nanoparticles within the biofilm, thereby disrupting it. Preferably, the method further comprises (d) pulsating the microbubble(s). Most preferably, the method comprises administering one or more antibiotics, which now have access into the biofilm.

The inflicted damage may also optionally enable other mechanisms to successfully attack the host bacterial colony. For example, the damage may enable antibiotics, immune system cells, and/or bacteriophages access into the damaged biofilm, contaminate its host bacteria and in turn, inducing at least partial killing of the bacterial population. Alternatively, effective treatment may be conducted by co-administering of bacteriophages and antibiotics to the contaminated tissue following treatment by the methods of the present invention. Other treatments are described in greater detail below.

### Materials for Biofilm Treatment.

According to optional but preferred embodiments of the present invention, one or more antibiotics are preferably administered to a subject in combination with treatment with nanoparticles as described herein. One or more optimized antibiotics are preferably administered to a subject to enhance or complete the treatment methods provided by the present invention.

The administered antibiotics may optionally and preferably include but are not limited to one or more compositions from the following groups: the quinolones group which includes: nalidixic acid, cinoxacin, norfloxacin, ciprofloxacin, sparfloxacin, and the like; anti-urinary tract infections group which includes: ethenamine, nitrofurantoin and the like; beta.-lactam antibiotics group which include penicillins, cephalosporins and the like; the penicillins sub-group which includes penicillin G, penicillin V, methicillin, oxacillin, cloxacillin, dicloxacillin, nafcillin, ampicillin, amoxicillin, and the like; the cephalosporins group which compounds such as cephalothin, cefazolin, cephalexin, cefadroxil, cefamandole, cefoxitin, cefaclor, ceftazidime, and the like; the aminoglycosides group which include: streptomycin, gentamicin, tobramycin, and the like; and the related antibacterial agents group which includes: chloramphenicol, clindamycin, spectinomycin, vancomycin, bacitracin, and the like; the macrolides group which includes erythromycin, clarithromycin, and the like.

The present invention, in some embodiments, optionally and preferably encompasses the use of materials and compositions instead or in addition to antibiotics in order to complete or enhance the treatment including but not limited to bacteriophages, and immune system stimulants. These materials may benefit the damage inflicted to the biofilm due to the pulsating microbubbles effects as described above. For example, bacteriophages (often known simply as “phages”) are viruses that grow within bacteria. The name translates as “eaters of bacteria” and reflects the fact that as they grow most bacteriophages kill bacteria. However, many types of biofilms protect the host bacteria against attack of specific bacteriophages which attack them and hence the biofilm must first be disrupted before a successful attack.

One or more of such materials may be selected from the following groups: bacteriophages including but not limited to: a wide spectrum phage, a phage effective against multiple species of specific bacteria, a phage effective against other bacteria genera, a phage effective against narrow range of species of a specific bacteria type, a phage comprising a heterologous gene encoding a polysaccharide lyase enzyme, a phage from any of the groups described by Sharp in US patent application 20060140911. Soothill et. al., in US Patent application 20070190033 suggested to use specific bacteriophages for attacking P. aeroginosa bacteria which build a biofilm. These bacteriophages may also optionally be used.

Materials for stimulating the immune system include but are not limited to: materials for activating T-cells, weakened bacteria, weakened toxins associated with bacterial activity, activated T-cells, etc.

Biofilm component lysing enzymes include but are not limited to: Alginate lysing enzyme, Dispersion B. etc.

### Formulation and Administration

According to some embodiments of the present invention, the absorbing nanoparticles may optionally be administered as a pharmaceutical composition which may optionally comprise antibiotics. Such compositions may be prepared by any method known in the art of pharmacy, for example by admixing the active ingredient with a carrier(s), diluent (s) or excipient(s) under sterile conditions.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions).

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils. Pharmaceutical compositions adapted for rectal administration may be presented as suppositories or enemas. Pharmaceutical compositions adapted for administration by inhalation include fine particle dusts or mists which may be generated by means of various types of metered dose pressurized aerosols, nebulizers or insufflators.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents.

### EXAMPLE 2

### Biofilm Removal from Surfaces of Non-Living Material

This example relates to removing biofilms from non-living materials, such as the surfaces of artificial implants and industrial surfaces, and the like.

It is widely recognized that the bacteria within a biofilm that form on a medical implant are much more resistant to antibiotics than their planktonic counterparts. Despite advances in sterilization and aseptic procedures as well as advances in biomaterials, bacterial and other microbial infection remains a serious issue in the use of medical implants. Bacterial infection of such devices, typically as biofilm, can in some cases necessitate the removal of implant from the patient body. The biofilm related implant infections can also result in illness, long hospital stays, or even death. A method for removal of biofilm formed on the surfaces of these medical is therefore highly desired.

Methods based on sonication, vortexing, scraping, rolling, flushing, rinsing, brushing, and brushing have been used to dislodge microbial biofilms from a variety of surfaces, both of medical devices and non-medical abiotic surfaces. Since most surfaces contain microcavities which are hard to access, most mechanical methods, with or without application of sonic energy showed only partial effectiveness as methods for biofilm removal.

Trampuz et al, in US patent application 20050241668 suggested extracting planktonic bacteria from contaminated surfaces using an ultrasonic bath comprising suspension of encapsulated microbubbles. However this method is not practical for in-vivo implants. Zimmeris et. al., in US Patent Application 20050268921 have suggested that exposure of implant surfaces to vibrations with nanometer scale amplitude prevents biofilm growth on it at close proximity. However, using this method requires an internal vibration source fed from an electrical supply, or mechanical coupling between the surface and a separate vibration source.

Exposing absorbing nanoparticles fixated on a surface suitable for its fixation to electromagnetic radiation and ultrasound sufficient for generating a nucleation bubble, results in a nucleation bubble adjacent to the absorbing nanoparticle. The hard surface may dictate asymmetric expansion pattern of the nucleation bubble. Further exposure of the nanoparticle to ultrasound may evolve the nucleation bubble into microbubble with asymmetric expansion pattern. Such microbubble will pulsate with the localized ultrasound amplitude. Exposing the nanoparticle partially covered with biofilm to electromagnetic radiation and ultrasound may induce various effects on the biofilm, such as: reduced adhesion of biofilm to the surface, enhanced access of antibiotics into the biofilm, complete removal of biofilm from surface and dispersion of the biofilm due to host bacteria induced processes. Clearly, each of these processes may enhance antibiotics ability to eradicate the biofilm host bacteria.

According to preferred embodiments of the present invention there are optionally and preferably provided methods for disrupting or at least reducing the protection ability of biofilm attached to an implant surface which is seeded with absorbing nanoparticles. Such implants optionally and preferably include but are not limited to intracorporeal or extracorporeal devices (e.g., catheters), temporary or permanent implants, stents, vascular grafts, anastomotic devices, aneurysm repair devices, embolic devices, and implantable devices (e.g., orthopedic or dental implants) and other types of implants, as listed by Hunter et al., in US Patent application 20060127438, hereby incorporated by reference as if fully set forth herein.

According to preferred embodiments of the present invention, the implants include implants which are removable and/or which project outside the body, such as a catheter for example.

Preferably, the surface to be treated comprises a coating layer suitable for fixating nanoparticles, on which fixated absorbing nanoparticles are present. Preferably, the absorbing nanoparticles are arranged in clusters and the clusters comprise between 5 and 50 in each cluster. Preferably, the average inter-nanoparticle distance ranges between 0.1 and 3 microns. Preferably, the absorbing nanoparticle structure supports anchoring to the layer and generation of nucleation at its exposed section. Preferably, the layer thickness is at least twice the equivalent diameter of the absorbing nanoparticles.

Optionally and more preferably the coating layer has thermal insulation properties and most preferably thermal conductivity similar or lower than of water. Preferably, the coating layer is made of ceramic materials or equivalent so as to maintain the attachment of absorbing nanoparticles after repeated generation of nucleation sites.

In other aspects, the coating layer and the implant structure do not interfere with the generation of enhanced electric field at close proximity to the absorbing nanoparticles, during exposure to microwave radiation. Preferably, the coating layer comprises material selected for enhancing the generation of nucleation bubbles following exposure to microwave radiation.

In preferred embodiments of the present invention, the absorbing nanoparticles may be integrated into the coating and the coating structure may be porous so as to enable generation of nucleation site within the coating layer.

In other embodiments, the absorbing nanoparticles may optionally and preferably be printed, preferably as clusters onto the coating layer using printable jet droplets. In other aspects the coating material may be mixed with absorbing nanoparticles and may be activated by selective removal of matrix material and exposure of absorbing nanoparticles following stabilization.

In yet other embodiments, the coating layer may optionally and preferably be applied as a paste, spray, printable jet droplets or sol-gel based material. Its stabilization may be achieved through natural drying, light curing, heat curing, drying, chemical reaction, or any other suitable process that enable fixation of the nanoparticles to the coating. The coating functionalization for the designated application may be achieved through plasma cleaning, wet or dry chemical reaction, and other suitable processes.

By way of illustration, FIG. 4 describes one exemplary, illustrative but preferred embodiment of a system for bacteria removal from liquid immersed protected surface. Stage I depicts a detailed view of a protected surface 400 contaminated with at least one biofilm 405 structure comprising a bacterial colony 410 in a polysaccharide envelope 455 and secrated by internal memeranges 460. The protected surface 400 is preferably coated with absorbing nanoparticles 415 which are in contact with the liquid. An electromagnetic source 425 is preferably operable to expose the biofilm 405 to a period of electromagnetic radiation 430. An ultrasound source 435 is optionally and preferably operable to expose the biofilm 405 to ultrasound energy 440.

At stage I, the protected surface is first preferably treated with antibiotics 420, exposing the absorbing nanoparticles 405 (or a cluster thereof) on the protected surface 400 to the electromagnetic radiation 430 and ultrasound energy 440 generate a microbubble 450 is generated in close proximity to at least a portion of nanoparticles 405 (or clusters thereof) in the process described above. Multiple microbubbles 450 evolve through interaction with ultrasound energy 440 and pulsate at the ultrasound frequency (stage II). The pulsating microbubbles induces local microstreaming which apply sufficient shear stress on the biofilm 405 to damage internal membranes 460 and adhesion to the protected surface 400 (stage III). Finally, the overall biofilm 405 damage and the microstreaming due to the pulsating microbubbles 450 enables antibiotics 420 access to at least a portion of the bacterial colony 410 within the damaged biofilm 405, thereby enabling a certain level of destruction of the bacterial colony 410 and at least a portion of the released planktonic bacteria 475 (stage IV).

In preferred embodiments of the present invention, the pulsed electromagnetic energy is preferably brought directly to the protected surface(s). In certain aspects, the pulsed electromagnetic source is a photonic source and its energy may be brought to the protected surface(s) by light guides equipped with a dispersive fiber optics applicator. In yet other aspects, the pulsed electromagnetic source is RF source and its energy may be brought to the protected surface(s) by a waveguide equipped with matching terminating emitter, a metal wire structure, or an RF applicator, such as the device described by Kopti et al [11].

Much like implants, surfaces of objects used to handle or store food products and raw materials and water are prone to development of biofilms. Other types of surfaces include industrial hard to access surfaces with frequent or continuous contact with water or aqueous solutions such as of closed water circuits, e.g., air conditioning systems, water reservoirs etc. Such surfaces are prone to developing biofilm comprising dangerous bacteria such legionnaire disease bacteria. All of these different surfaces may be treated according to the above methods of the present invention.

### REFS

[a1] Medical Biofilms, Module 7, sections 4, 5 http://www.erc.montana.edu/biofilmbook/MODULE_{—}07/Mod07_S05_Blue.htm (2007).
[a2] Med-Market Diligence, “World-wide wound management 2002-2012, Products, Technologies & Markets opportunities,” Report S200 (February 2003).
[a3] A. Marra, “Can Virulance factors be viable antibacterial targets?.” Expert. Rev. Anti-infective Ther. V 2 n 1 p. 62-71 (2004).
[a4] Biotech Finances, “Research and Markets: Antibiotics and Drug Resistance 2007—Drug Innovation and the Strategy to Combat Antibiotic Resistance Mechanisms,” August 2007
[a5] From Knocking Bacteria Out of Biofilms to Piggybacking Gene Therapy on Microbubbles”, Mayo clinic—Discovery edge (1998).
[a6] M. Odonnell, L. Balogh et al, “Colloid loaded dendrimers for fighting cancer,” Nanotech Conf. May 24, 2007.
[a7] S. Bakhdi and J. Tranum-Jensen, “Alpha toxin of Staphylococcus aureus., Microbiological review, v 55 n 4 p. 733-7451 (1991).
[a8] M. S. Longuet-Higgins, “Viscous streaming from an oscillating spherical bubble,” Proc. Roy. Soc. Lond. A V 454 p. D725-D742, (1998).
[a9] P. Marmottant, S. Hilgenfeldt et. al., “CELL PERMEABILISATION AND TRANSPORT FOCUSED AROUND OSCILLATING MICROBUBBLES,” XXI ICTAM, Warsaw, Poland, p. 15-21 (August 2004).
[a10] C. Uhlemann, B. Heinig, et. al., “Therapeutic ultrasound in lower extremity wound management,” Lower Extremity wounds V 2 n 3 p. 152-157 (2003).
[a11] Medical Biofilms, Module 7, sections 4, 5 http://www.erc.montana.edu/biofilmbook/MODULE_{—}07/Mod07_S05_Blue.htm (2007).
[a12] J. Wu, “Temperature rise generated by ultrasound in the presence of contrast agent,” Ultrasound Med. Biol. V 24 n 2 pp. 267-274 (1998).
[a13] Z. Hazan, J. Zumeris, et. al., Effective prevention of microbial biofilm formation on medical devices by low energy surface acoustic waves, “Antimicrob. Agents Chemother. V 50 n 12 p. 4144-4152 (2006).
[a14] T. K. Lu and J. J. Collins “Dispersing biofilms with engineered enzymatic bacteriophage,” PNAS V 104 n 27 p. 11197-11202 (2007).
[a15] L. R., Hirsch, R. J. Stafford, et al., Nanoshell-mediated near-infrared thermal therapy of tumors under magnetic resonance guidance,” PNAS v 100 n 23 pp. 13549-13554 (2003).

### Section 3—Applications of Microbubbles for Localized Drug Release

There are a range of methods of delivery of an agent to a subject. For in vivo administration, methods include catheters, injection, scarification, etc. However, many of these methods are systemic, or at best regional in application. This can result in delivery of an agent to normal tissues, where the effect of the agent can be deleterious. Thus, a method for targeted delivery of an agent to only a particular region would be desirable. It would also be desirable to do this in as non-invasive a manner as possible. Accordingly, localized targeted drug delivery is highly desirable for a wide array of applications.

A key advantage of localized drug release is the ability to increase drug concentrations locally while avoiding side effects that usually are associated with systemic delivery. Higher drug concentrations at the treatment site enable improved drug penetration into the treated tissue. Localized drug release becomes critical, in cases where the effective drug concentration required to treat a specific diseased tissue, is beyond the upper limit of that drug concentration, as dictated by the allowed toxicity level to healthy adjacent tissue. Another example is drugs with short half-lives which add to the criticality of transporting the therapeutic molecules to the target cells as quickly as possible.

Localized drug release is particularly effective in treating conditions, such as some cancers, in which the rate of cell division or migration is high. In such conditions, the time for the therapeutic molecules to reach the cancer cells from their release site is critical. The molecules must reach the target cells, which may have migrated deep into the healthy tissue, in sufficient volume and concentration and at a rate that will enable them to attack the cells with a therapeutically effective dosage.

A good example for the benefit of localized drug delivery is cancer treatment using chemotherapeutics with high toxicity, such as doxorubicin for example. These drugs could not be regularly administered for cancer therapy because of toxic effects for normal tissues such as bone marrow, gastrointestinal tract and hair follicles. Side effects that occur as a result of toxicities to normal tissues mean that anticancer chemotherapeutic drugs are often given at sub-optimal doses, resulting in the eventual failure of therapy, often accompanied by the development of drug resistance and metastatic disease.

The simplest route of localized drug delivery is local administration to the diseased tissue or organ. Although advantageous over systemic delivery, the dependence of local drug administration on the naturally occurring passive diffusion process falls short of addressing the need for deeper penetration of molecules into the tissue.

Thus, effective modern localized drug release comprises (a) Administering drug encapsulated in suitable particles comprising the desired drug composition to patient or patient region; (b) accumulating the particles near the target cell, or tissue and (c) triggering the release of the drug from the particles by an extracorporeal energy source.

A drug carrying particle operable for localized drug release should have at least one or more of the following properties: stability against biodegradation during its period of presence in the circulation; tendency for accumulation near the targeted cells or tissue, either by suitable ligand or adopted encapsulation properties; appropriate coating or material to prevent internalization by cells of the immune system; release of carried drug at tolerable stimuli (e.g., heating not beyond 41 C); resistant to accidental release by physiological pressures; minimal spread of threshold value for drug release (e.g., overpressure, temperature); an encapsulation shell which enables penetration through blood vessels (e.g, liposomes); and/or a small size for easy penetration through the interstitium (e.g., below 500 nm); or a combination of any of the above.

There are several ways to accumulate particles within the targeted tissue.

For example, the drug can be encapsulated in a macromolecular carrier, such as a liposome. In turn the volume of particle distribution within the patient body is significantly reduced and the local concentration of drug in the tumor area is increased (Drummond et al., 1999), resulting in decreases of dosage and nonspecific toxicities and increase the effectiveness of drug dosage.

The use of ligand-targeted particles is an effective method of accumulation particles near target cell, organ or tissue. For example, lipid shelled liquid fluorocarbon particles have been used to deliver therapeutic agents to cells selectively by binding to specific cellular epitopes (Lanza et al. (2002) Circulation 106:2842-2847). The particles are targeted by incorporation of selected ligands (e.g., monoclonal antibodies, small molecules, etc.) into the lipid membrane through, for example, bifunctional intermediaries complexed to lipid adducts that situate within the lipid membrane of the particle.

Previously developed formulations of liposomes were removed rapidly from blood circulation by the reticuloendothelial system (RES), thus preventing the liposomes from reaching the target sites. Liposomes containing various lipid derivatives of polyethylene glycol (PEG) have resulted in improved circulation time and tumor localization (Papahadjopoulos et al., 1991). More recent PEG coated liposomes such as Stealth® have demonstrated long circulation periods measured in 12 hours and more. This long circulation period enables significant long term accumulation in regions of high metabolic rate such as tumor tissue.

The release of carried drug from accumulated particles is typically induced by an extracorporeal energy source which provides heating, mechanical, or electrical energy for rupture of the particle or inducing leaks in it. Common energy sources used for localized drug delivery include but are not limited to ultrasound, RF, light electrical energy or combination thereof. Ultrasound energy is the most commonly used energy source for drug delivery, since it also enhances the rate and depth of transport of therapeutic substances through the treated tissue.

Recently, drug carrying nanocapsules [a6] have been suggested as method for fighting cancer after rupture by therapeutic ultrasound. Nanocapsules with a size of several hundred nanometers have better migration properties compared to contrast agent microbubbles. In principle such nanocapsules with suitable ligands may attach to biofilm structure serve as nucleation sites for microbubbles production. However, their size severely limits their migration rate through blood vessels and especially through the intercellular space.

Joyce in US patent application 20050214356 suggested the use of vesicles comprising a nanotube within the vesicle or within its shell for localized drug release. His suggested mechanism based on the nanotubes includes: pore forming agents suitable for lysis of liposome are released from the nanotubes following exposure to external energy source; increase in energy state such as heating of suitable nanoparticles by exposure to external energy source.

One approach which has been taken for localized delivery of therapeutic compounds is the use of gaseous precursor-filled microspheres, as described for example in U.S. Pat. No. 6,443,898. In this system, the gas in the microspheres expands when the microspheres are heated to a certain temperature, rupturing the microsphere and releasing the compounds contained within. Ultrasound, microwaves, magnetic induction oscillating energy, and light energy can be used to raise temperatures in a localized manner to rupture the microspheres. However, this system is associated with several important disadvantages, including the size of the microspheres, which typically have a diameter in the range of microns rather than nanometers. Such a large size restricts the utility of this method. In addition, the walls of the microspheres are typically comprised of lipids and/or polymers. Particularly considering their size, the microspheres are not readily available to modifications which could allow them to be transported through blood vessels, tissue or barriers.

However, it is well known that sub micrometer particles (i.e. nanocapsules) and especially liposomes, exhibit certain degree of transport through blood vessels, barriers and through the interstitium see for example in [a6]. The transport rate is accelerated by ultrasound and further accelerated by ultrasound in presence of microbubbles (see for example Heart et al. in US Patent application 20060058708).

The release of drug from particles by ultrasound induced rupture typically requires intense ultrasound flux, such as that generated by HIFU. A modern HIFU source for controlled particle rupture has important advantages: First, it can focus intense ultrasound energy within the desired region, leaving the surrounding tissue mostly unheated. Second, such sources are equipped with features which can control the intensity of ultrasound within the focal region. However, as noted above, the use of HIFU for localized rupture of particles of the present invention has a number of safety issues. Typical intensities suitable for ultrasound induced ruptures of drug carrying particles are in the range of 6-60 W/cm2 or MI between 0.5 and 1.5. In general, the MI permitted for diagnostic ultrasound is below 1.9. Still, exposing tissue in many regions of patients to ultrasound whose MI is larger than 1 to 2 may result in blood vessel rupture due to cavitation, ischemia, and even irreversible damage to cells due to hyperthermia. Thus, localized drug release treatment based on a HIFU source operating well above MI=1 is conducted slowly within small regions and requires special FDA approval, and even then must be performed under continuous control of high cost imaging instrument such as MRI.

Due to the above, in many applications it is required that the peak ultrasound pressure is below 1 MPa and in some cases below 0.3 MPa. Many phenomena have been recently related to the application of intense ultrasound: These include local ischemia, local internal bleeding and excessive kill of sensitive cells such as T-cells. Without wishing to be limited by a single hypothesis, methods for reducing the ultrasound threshold required for drug release from particles are limited due to two factors: (a) uncontrolled rupture of particles in regions with enhanced ultrasound power density far from the focal zone of the ultrasound beam; and (b) preventing accidental release by setting the threshold beyond the level which may occur within normal physiological activity. For example, normal physiological pressures include those pressures encountered in vivo, including pressures within the heart and arteries, as well as compressive pressures of passing through constrictions such as arterioles.

The ultrasound rupture threshold of a particular particle varies with the shell thickness, diameter and strength and the ultrasound frequency. The threshold for specific shelled particles is typically specified by the Mechanical Index (MI). Typical MI for rupturing shelled microbubbles ranges between 0.5 and 1.5 (see for example Heart et al. in US Patent application 20060058708) which corresponds with ultrasound power density of 6 to 60 W/cm2 in pure water at 1 MHz.

One possible way to reduce the MI is by narrowing the spread of the particle geometry, which also minimizes the chance of accidental release. The ultrasound overpressure required for rupture of a particular particle varies with the shell thickness and strength. Conston in U.S. Pat. No. 6,896,659 teaches a method for controlled rupture of drug carrying microcapsules by keeping a constant ratio between their shell wall thickness and shell diameter. Indeed, the rupture pressure, P.sub.max, for a particle comprising a thin hollow spherical shell is:

$${P}_{max} = 4 {σ}_{t} \frac{h}{D} \qquad ( 5 )$$

Here P.sub.max is the maximum difference between the pressure of the composition inside the particle and the minimal local pressure, h/D is the shell wall thickness to shell diameter ratio and sigma.sub.t is the tensile strength of the wall material.

When the particle is exposed to ultrasound field, P.sub.max is expressed as

Pₘₐₓ=P_{comp}−Pₐ+Pᵤₗₛ (6)

Where P.sub.comp is the pressure of the composition inside the particle, P.sub.a is the ambient blood pressure and P.sub.uls is the maximal rarefaction (negative) pressure of the propagated ultrasound field.

As mentioned above, safety considerations require that particles generating gas bubbles should be ruptured so that P.sub.max values are significantly higher than the right hand side of Eqn. (6) under all blood pressure and temperature scenarios. This safety margin is essential for minimizing the chance for accidental rupture and following exposure to the ultrasound beam margin. In preferred embodiments of the present invention, the nominal P.sub.max is at least 0.05 MPa higher than the right hand side of equation (6) above, at 37 C.

Kane in US Patent application 20060057192 suggested a method for localized delivery of bioactive composition to a cell, comprising the steps of: (a) Administering heat sensitive particles comprising a bioactive composition, to desired region of a patient; (b) Exposing the region to ultrasound with sufficient intensity so as to locally heat the tissue in the region to suitable temperatures. (c) Inducing thermal heating of the particle vicinity so as to induce release of the bioactive composition to a cell.

Kane suggested heat responsive shell materials such as liposomes with transition temperature of 41 C. The localized heating may be achieved by focused ultrasound or focused RF radiation. Microwaves are another alternative to ultrasound for transcranial and deep energy deposition; however penetrating wavelengths in this domain cannot be focused as well as ultrasound, thereby limiting the ability to localize the drug release.

De Zwart et. al., [1] have demonstrated that drug release from heat sensitive liposomes requires heating of target region to about 43 C for several tens of seconds. Such heating level and exposure period requires ultrasound overpressures of 1.5 MPa [2] which corresponds with 20 W/cm2, typically attained by HIFU sources with their limitations as described above.

Photolytic uncaging has also been used for localized drug delivery. The science of photolyctic uncaging, i.e., drug release from particles following photo-disintegration of its shell, is another method for releasing biologically active agents in spatially and temporally restricted tissue region. This method relies on photonic energy as its focused deposition method. Unfortunately, the only wavelengths applicable to this process not strongly absorbed by some endogenous molecules are near-infrared and microwaves.

West et al. in U.S. Pat. No. 6,513,944 teaches method for localized drug release by using particle comprising light absorbing nanoparticles whose absorption line can be tuned to the near infra-red. These nanoparticles provide localized drug release by specifically heating the heat sensitive shell, inducing leak from the encapsulated liquid. Another way to rupture particles is to expose nanoparticles seeded shelled particles to laser pulse. This method has been demonstrated at energy density of 50 mJ/cm2. However, NIR radiation induced drug release methods suffer from a common limitation: although near-infrared can penetrate into tissue between 1 and 5 centimeters, it is impossible to focus due to a severe scattering affect.

According to preferred embodiments of the present invention, there is preferably provided a method which overcomes the above drawbacks of the background art, by generating at least one nucleation bubble within a particle comprising a bioactive composition for localized release of the bioactive composition, preferably at or within a specific region or location in the subject. More preferably, the particle comprises a bioactive composition, volatile liquid, and absorbing nanoparticles operable for inducing drug delivery when exposed to suitable electromagnetic and ultrasound radiation. The size of particle may optionally and preferably range between 200 nm and 10 micron. Additionally, the particle may optionally comprise other nanoparticles with suitable attached ligands which promote their attachment to cells or tissue, radioactive material, viruses, emulsion, liposomes etc.

The particle may optionally be administered systemically, by an implant, by injection, orally, intramuscularly, intrathecally or by any other suitable administering procedure. Any standard method can be applied for administering the particles to the cells or tissue as is known in the art.

Preferably, localized drug release from a particle is conducted by simultaneous exposure of the particle comprising absorbing nanoparticle(s) to electromagnetic radiation and ultrasound for turning the nucleation bubble into a microbubble within the particle. When the microbubble reaches the critical radius, the volatile liquid content within particle evaporates spontaneously, followed by rupture of the particle. The volatile liquid may be a halocarbon, water or any liquid composition that can be held in liquid state within a particle.

According to some embodiments, at least one cluster of absorbing nanoparticles may optionally be used for reducing the ultrasound threshold for localized drug delivery from particles, while minimizing accidental release of drug in regions adjacent to the target region. Reducing the ultrasound threshold is preferably attained by methods described above.

In a preferred embodiment of the invention, there is provided a method for localized delivery of a therapeutic or bioactive composition from a particle, comprising delivering a particle comprising bioactive composition, absorbing nanoparticles and volatile composition to cells or tissue; and exposing the particle to simultaneous electromagnetic radiation beam and ultrasound radiation to induce release of the bioactive composition. More preferably, the method further comprises: generating a microbubble within the particle sufficient to evaporate at least a fraction of the volatile composition; and breaching the particle due to enhanced internal pressure, thereby causing release of its bioactive content to the cells or tissue.

In other aspects, the electromagnetic radiation and ultrasound energy induce a microbubble within the particle. However, its peak radius is below the evaporative rupture threshold, Still, the increased microbubble volume and its pulsations induce stress on the shelled particle, and may result in rupture of the particle when the wall stress exceeds the encapsulation tensile strength. Thus, the particle could be used for drug release without complete evaporation of its content.

Similarly, simultaneous exposure of a pro-permeable shelled particle filled with volatile composition mixed with absorbing nanoparticles and bioactive composition, to electromagnetic pulses and ultrasound would generate a vapor filled microbubble, preferably at close proximity to the particle shell, through the process described hereinabove. Once the microbubble grows beyond a certain radius, its coupling efficiency with the ultrasound radiation increases and its pulsation level (long to short ellipsoid diameters) becomes significant. In turn, the microbubble pulsations induce significant permeability in the particle resulting in leakage of its bioactive composition. The resultant permeability is attained at much lower ultrasound overpressures compared to typical levels required for particle sonoporation.

In yet another optional embodiment, there is provided a method for localized delivery of therapeutic or bioactive composition from a particle, comprising delivering a particle comprising bioactive composition, absorbing nanoparticles and pro-permeable membrane wall and an attached ligand suitable for attachment to a targeted cell, to the eye; contacting the particle to selected ocular target cells using a suitable ligand; and exposing the particle simultaneous electromagnetic radiation beam and ultrasound radiation. More preferably, the method further comprises generating a microbubble near inner wall of the particle; and inducing permeability of the membrane shell due to pulsation of the microbubble, in turn enabling enhanced transport of the bioactive compositions from the particle to the targeted cells or tissue.

By way of illustration, FIG. 5 describes one exemplary, illustrative but preferred method for localized drug release in accordance to the present invention. In reference to FIG. 5A, a particle 520 operable for localized drug release to targeted cells 510 within a localized region of a patient, optionally and preferably comprises a breakable shell 525, absorbing nanoparticles 530, preferably arranged in a cluster, immersed in a liquid mixture 540 comprising volatile composition and suitable bioactive composition suitable for inducing therapeutic effect in cell 510. The particle shell 525 is preferably equipped with one or more ligands 545 suitable for attachment to the targeted cells 510. Following administration to the target region of a patient, particle 520 comes into close proximity with targeted cell 510 by attachment of ligand 545 to a matching receptor 550 which is expressed at a sufficiently high level on the targeted cell membrane 555.

In reference to FIG. 5B, the particle 520 attached to the targeted cell 510 is preferably exposed to a period of electromagnetic radiation 560 and ultrasound radiation 565 acting on the relevant region of the patient. The combined action of electromagnetic beam 560 and ultrasound radiation 565 generates a microbubble 570 within the liquid mixture 540. During the growth of microbubble 520, vapor of volatile liquid from the liquid mixture 540 increase its volume by rectified diffusion process. Preferably, the liquid mixture 540 temperature does not exceed the boiling temperature of the volatile liquid at the ambient pressure.

In reference to FIG. 5C, when reaching a size of 1-2 micron, the intensity of the microbubble 520 pulsations 568 is significantly enhanced due to the ultrasound radiation 565, inducing pressure pulsations in the shell 525. After a short period, the shear forces and pressure pulsations within the liquid mixture 540 induce a break 570 in the particle shell 525. The break 570 in the particle shell 525 enables flow of the liquid mixture 540 through the particle shell 525, creating a spill volume 575 comprising significant concentrations of the bioactive composition in close proximity to the targeted cell 510, thereby inducing the therapeutic effect 580 in targeted cell 510 from the action of the therapeutic components.

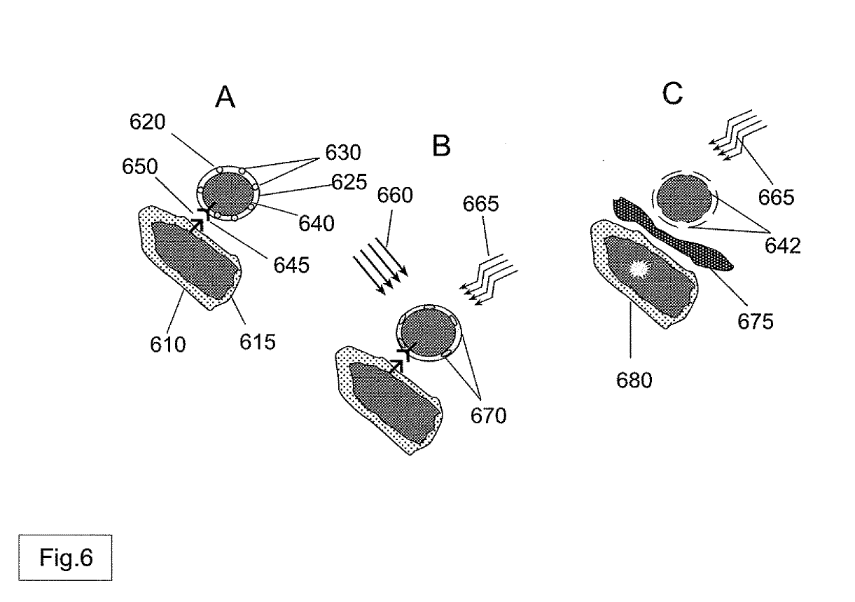

FIG. 6 describes one exemplary, illustrative but preferred method for localized drug release in accordance to the present invention. In reference to FIG. 6A, a particle 620 operable for localized drug release to targeted cell 610 within a localized region of a patient optionally and preferably includes a shell 625 and a liquid mixture 640, comprising a volatile composition and suitable bioactive composition. The shell 625 comprises integrated absorbing nanoparticles 630, preferably arranged in clusters, and a fraction of the liquid mixture 640. The particle shell 625 is equipped with one or more ligands 675 suitable for attachment to the targeted cells 610. Following administering to the target region of a patient, particle 620 comes into close proximity with targeted cells or tissue 610 by attachment of ligand 645 to a matching receptor 650 which is expressed on the targeted cell membrane 655.

In reference to FIG. 6B, the particle 620 attached to the targeted cell 610 is preferably exposed to a period of electromagnetic radiation 660 and ultrasound radiation 665 acting on the region of patient. The combined action of electromagnetic beam 660 and ultrasound radiation 665 generates multiple nanobubbles 670 within the liquid mixture 640. During growth of nanobubbles 670, the vapor of volatile liquid from the liquid mixture 640 within shell 625 increases their volume by rectified diffusion process. Preferably the temperature of the liquid mixture 640 within the particle 620 may not exceed the boiling temperature of the volatile liquid at ambient pressure.

In reference to FIG. 6C, when the nanobubbles 670 within the shell 625 reach a preferred size of about 200 nm, the shear forces and pressure pulsations within the liquid mixture 640 induce at least one discontinuity 642 in the particle shell 625. The discontinuity 672 enables flow of the liquid mixture 640 through the particle shell 625, creating a spill volume 650 comprising large concentrations of the bioactive composition in close proximity to targeted cell 610. The contact of the bioactive composition spill volume 675 with the targeted cell membrane 615 induces a therapeutic stimulus 680 in the targeted cell 610.

In yet another preferred embodiment of the invention, there is optionally and preferably provided a method for localized delivery of therapeutic or bioactive composition from a particle to specific cells or tissue, comprising administering to the cells or tissue a mixture comprising: (i) at least one particle comprising bioactive composition, and (ii) absorbing nanoparticles operable for attachment to the cells or tissue; and exposing the cells or tissue to simultaneous electromagnetic radiation and ultrasound radiation. Preferably microbubbles are generated near the nanoparticles within and/or at close proximity to the cells or tissue them to selectively couple ultrasound energy so as to heat the cells or tissue. Next, the particle ruptures under the combined effect of elevated surrounding temperature and ultrasound, in turn releasing its bioactive content to the cells or tissue.

The ultrasound parameters depend upon many variables including the particle size, shell thickness, type of carried or shell integrated absorbing nanoparticles, volatile liquid properties, etc. In preferred embodiments, the particles will be designed such that the peak ultrasound pressure will be below 1 MPa and the preferred mechanical index between 1.0 and 0.2 and preferably between 0.3 and 0.5. Short period of ultrasound are preferred between 200 and 1 second and more preferably between 60 and 5 seconds. Particles of the present invention of the liposome whose typical size is about 100 nm, type may require higher MI since they carry only a few absorbing nanoparticles.

The preferred microwave parameters would be frequencies between 100 MHz and 3 GHz, and total microwave dose below 50 J/cm.sup.2 and more preferred between 20 and 1 J/cm.sup.2. In other aspects, the microwave radiation should be pulsed with preferred pulse width between 10 mili second and 1 microsecond.

### Materials for Localized Drug Release

According to preferred embodiments, the present invention preferably encompasses the use of liquid filled particles for delivery of therapeutic composition to a tissue. In other aspects, the liquid filled particles may optionally and preferably contain a suitable bioactive composition to be delivered to predetermined cells or tissue. The particle size may preferably range from about 100 nm to about 3 microns. The particle may comprise a lipid shell, polymer shell, protein shell and combination thereof. The particle may also optionally comprise a volatile liquid so as to enable release of the bioactive composition to the desired object. The particle is preferably optimized for transport through the vasculature or within tissue, with or without the help of an extracorporeal energy source. The particle is also preferably designed for optimal lifetime in the circulation and to avoid the RES.

The particle may comprise: bioactive compositions, volatile liquid, absorbing nanoparticles, nanoparticles with ligands which promote attachment to cells or tissue, radioactive material, viruses, emulsion, small size liposomes, etc.

The content of the particles suitable for localized drug delivery optionally and preferably include but are not limited to one or more therapeutic agents comprising drug substances, small chemical molecules, proteins, polypeptides, oligonucleosides, nuclear enzymes, DNA plasmids, and polymers, inside the intravesicular space. The volatile sterile liquid may optionally comprise one or more composition including but not limited to: ocular compatible fluorocarbon, water, ethanol, polyol (including iso-propanol) etc.

The particle may optionally and preferably comprise a shell selected from the group: a lipid shell, polymer shell, protein shell and combination thereof. The particle shell and internal construction is preferably constructed of “pharmaceutically accepted” materials. The thickness of the particle shell will be determined in n part by the mechanical properties of the shell and by the mechanism by which the particle ruptures, becomes leaky or releases its carried drug. The shell thickness should be sufficient to prevent particle rupture due to physiological conditions according to the particle diameter. The shell thickness of drug carrying particles suitable for vascular applications shell thickness will be in the range from about 25 nm to about 1000 nm.

In many drug delivery applications, it is important that the particles circulate through the capillary network unimpeded. For such instances, particle diameter should be preferably in the range of 1 to 10 microns. In cases where extravasation is required the particles diameter may range between 100 nm and 3 micron and preferably between 200 nm and 1 micron.

The present invention, in some embodiments, optionally and preferably encompasses the use of lipid encapsulated particles for localized release of bioactive composition. The particle may optionally and preferably be constituted, for example, by an emulsion or liposome which comprises volatile liquid and absorbing nanoparticles. In a specific example, the lipid encapsulated particles may optionally be constituted by a perfluorocarbon emulsion, the emulsion having incorporated into their outer coating a lipid compatible moiety such as a derivatized natural or synthetic phospholipid, a fatty acid, cholesterol, lipolipid, sphingomyelin, tocopherol, glucolipid, stearylamine, cardiolipin, a lipid with ether or ester linked fatty acids or a polymerized lipid.

In certain aspects, the lipid shelled liquid filled particles are liposomes. Liposome carriers, hereinafter “liposomes” are microscopic carriers that consist of one or more lipid bilayers surrounding aqueous compartments (see, generally, Bakker-Woudenberg et al., Eur. J. Clin. Microbiol. Infect. Dis. 12 (Suppl. 1):S61 (1993), Kim, Drugs 46:618 (1993). Liposomes protect the drugs from being metabolized and inactivated in plasma. Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 .mu.m to greater than 10 .mu.m. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Liposomes generally comprise lipid materials including lecithin and sterols, egg phosphatidyl choline, egg phosphatidic acid, cholesterol and alpha-tocopherol.

After intravenous administration, small unmodified liposomes (0.1 to 1.0 .mu.m) are typically taken up by cells of the reticuloendothelial system (RES), located principally in the liver and spleen, whereas liposomes larger than 3.0 .mu.m are deposited in the lung. In preferred embodiments of the present invention, an extended circulation time is needed for liposomes to reach a target region, for example, when liposomes are administered systemically.

In a preferred embodiment the liposomes are coated with a hydrophilic agent, for example, a coating of hydrophilic polymer chains such as polyethylene glycol (PEG) to extend the blood circulation lifetime of the liposomes (see, for example, Stealth Liposomes, CRC Press, Lasic, D. and Martin, F., eds., Boca Raton, Fla., (1995), and the cited references therein). In addition, incorporation of glycolipid- or polyethelene glycol-derivatized phospholipids into liposome membranes has been shown to result in a significantly reduced uptake by the RES (Allen et al., Biochim. Biophys. Acta 1068:133 (1991); Allen et al., Biochim. Biophys. Acta 1150:9 (1993)). Such surface-modified liposomes are commonly referred to as “long circulating” or “sterically stabilized” liposomes.

The absorbing nanoparticles carried by the liposomes and employed for drug release may be, for example, encapsulated in the aqueous interior of a liposome, interspersed within the lipid bilayer of a liposome, attached to a liposome via a linking molecule that is associated with both the liposome and the absorbing nanoparticle, entrapped in a liposome, complexed with a liposome, etc.

The present invention optionally and preferably encompasses the use of polymer and polymer associated lipid shelled particles. At least one absorbing nanoparticle may optionally be encapsulated in the liquid interior of a polymer shelled particle, entrapped in the polymer shell or organized within the particle. The polymer-lipid shell compositions may optionally be based on dextran, polysaccharide, anionic moieties in a salt polymer, etc. (see Bednarksi in US patent application 20040223911). The compositions optionally and preferably extend the circulation time of lipid shelled carriers, beyond those of PEG coated liposomes.

The particle carrying bioactive composition may optionally be encapsulated within an internal oil phase within an external aqueous phase, comprise a single phase liquid volume, protected by an internal polymer layer or cellular structure etc.

According to preferred embodiments, the present invention preferably encompasses the use of suitable ligands to achieve targeted delivery of bioactive composition by the attachment of suitable ligands to a particle carrying the bioactive composition. The present invention, in some embodiments, optionally and preferably encompasses the use of a ligand which may be, for example, constituted by (but not limited to) one or more of polysaccharides, monoclonal or polyclonal antibodies, viruses, receptor agonists and antagonists, antibody fragments, lectin, albumin, peptides, hormones, amino sugars, lipids, fatty acids, nucleic acids and cells prepared or isolated from natural or synthetic sources. In short, any site-specific ligand for any molecular epitope or receptor to be detected through the practice of the invention may optionally be utilized.

One or more ligands (either identical or several types) may optionally be conjugated to the particles directly or indirectly through intervening chemical groups such as an alkane spacer molecule or other hydrocarbon spacer.

The particle may be administered systemically, by an implant, by injection, or by any other suitable administering procedures. Any standard method can be applied for administering the particles to the cells or tissue.

The present invention, in some embodiments, optionally encompasses oral administration for delivery of particles for drug release. Particles may optionally be formulated in tablet, capsule, powder or liquid form. A tablet can include a solid carrier such as gelatin or an adjuvant. Liquid pharmaceutical compositions generally include a liquid carrier such as water, petroleum, animal or vegetable oils, or synthetic oil. Physiological saline solution, dextrose or other saccharide solution or glycols such as ethylene glycol, propylene glycol or PEG can be included.

The present invention encompasses the use of particles formulations suitable for injection. Particles may be formulated for intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium chloride injection, Ringer's injection, Lactated Ringer's injection. Preservatives, stabilizers, buffers, antioxidants and/or other additives can be included, as required.

### REFERENCES

[c1] D. P. O'Neala, L. R. Hirsch et. Al., “Photo-thermal tumor ablation in mice using near infrared-absorbing nanoparticles,” Cancer Letters v 209 p. 171-176 (2004).
[c2] J. Wu, “Temperature rise generated by ultrasound in the presence of contrast agents,” Ultrasound in Med. & Biol v 24 n 2 p. 267-274 (1998).

### Section 4—Applications of Microbubbles for Embolism Formation

Angiogenesis-dependent diseases (i.e., those diseases which require or induce vascular growth) represent a significant portion of all diseases for which medical treatment is sought. In certain of these clinical situations, (e.g., bleeding, tumor development) it is desirable to reduce or abolish the blood supply to an organ or region.

In many cases of tumor development, there is an abundance of blood vessels which are entering the tumor in all directions similar to the spokes of a wheel. The tumor induces the ingrowth of the host vasculature through the production of “angiogenic factors.” The tumor tissue expands distally along the blood vessels which supply it. Typically, the blood vessel density is greater in the vicinity of the tumor than it is in the surrounding normal tissue. Thus, embolizing the vasculature around a tumor would stop it growth and in turn suffocate it leading to its death.

Embolization may also be utilized as a primary mode of treatment for inoperable malignancies, in order to extend the survival time of patients with advanced disease. Embolization may produce a marked improvement in the quality of life of patients with malignant tumors by alleviating unpleasant symptoms such as bleeding, venous obstruction and tracheal compression, and humoral effects.

There are other clinical situations where it is desired to occlude blood vessels by embolization to treat conditions of excessive bleeding. For example, menorrhagia (excessive bleeding with menstruation) may be readily treated by embolization of uterine arteries. Arterial embolization may be accomplished in a variety of other conditions, including for example, for acute bleeding, vascular abnormalities, central nervous system disorders, and hypersplenism.

The traditional method for embolization of uterine blood vessels which feature excessive bleeding employs 100-300 micron spheres which are locally administered to large arterioles in order to occlude them. However, the spheres tend to wedge within these arterioles without providing efficient blocking. Accordingly, the effectiveness of this treatment to other angiogenic diseases is rather limited since only a fraction of the arterioles can be occluded. For example, the size of liver metastases may be temporarily decreased utilizing such methods, but tumors typically respond by causing the growth of new blood vessels into the tumor.

Carter et al., in US Patent Application 20060052701 suggested occluding the blood flow to the treated tissue by selectively treating specific portions of the nourishing vasculature with high intensity focused ultrasound (HIFU). The focused ultrasound radiation disrupts the small blood vessels in the targeted vasculature and blocks the blood flow through them. However, as mentioned above, the suggested treatment requires on-line imaging (e.g., by MRI) of the targeted region and bear safety issues regarding accidental break of disrupted blood vessel.

Hunter et al., in US Patent Application 20060127445 suggested administration of capsules carrying anti-angiogenic compositions such as paclitaxel in targeted vasculature system(s) so as to effectively occlude microtubules within the vasculature system(s). However administering a sufficient amount of the capsules for effective occlusion of the vasculature may embolize vasculature system nourishing healthy tissue as well, in the absence of an effective targeting mechanism.

Recently, Ye and Bull [d1] suggested using encapsulated gas bubbles for treatment of cancer and other angiogenesis-dependent diseases. Their approach is based on simultaneous occlusion of most blood vasculature which nourishes the targeted tissue. Capsules comprising encapsulated superheated fluorocarbon compositions are administered to the patient and accumulate in the vasculature system(s) which nourish the tumor. Ye suggested rupturing the capsules under High Intensity Focused Ultrasound (HIFU) irradiation thereby releasing the enclosed liquid which evaporates and generate fluorocarbon filled bubbles.

During the treatment the targeted vasculature, which is loaded with a sufficient amount of capsules, is scanned with HIFU source. Each exposed capsule rupture under the combined effect of internal pressure and ultrasound rarefaction pressure, thus releasing the superheated liquid which in turn generates a gas bubble sufficient to occlude arterioles. Typically, the generation of multiple gas bubbles in each targeted arteriole is required, until one of them wedges for example in a bifurcation and in turn, effectively occluding the arteriole. The resultant bubble “sausage” may stay a few days in the arteriole before disappearing. Ye claims that the suggested treatment results in effective blocking of the vasculature system(s) which nourish the tumor, leading to the death of the tumor. After the treatment, the fluorocarbon gas dissolves into the blood and is naturally removed from the circulatory system.

However, the gas bubble approach suffers from several drawbacks. Firstly, the generated gas bubbles may escape and travel in the blood system and in turn, may become lodged in the microcirculation, causing local ischemia. Further, extensive damage may be induced if the bubble lodges in the arterioles or capillaries in the brain or in the coronary circulations. Second, during evaporation, the released droplet may rupture or damage an arteriole, causing local internal bleeding. Also capsules may rupture in a vasculature nourishing healthy tissue adjacent to the treated vasculature, causing local ischemia, and fourth, HIFU scanning is considered hazardous treatment of internal tissue. Thus, HIFU scan must be employed at slow rate under diagnostic imaging control [d2]. Typically, very high cost MRI instrumentation is employed for diagnostic imaging during HIFU treatment.

The ultrasound assisted evaporation of volatile liquid from suspended liquid filled particles suitable for embolization is complicated, even if the liquid comprises superheated composition. Apparently, small superheated droplets of volatile liquids immersed in liquid do not evaporate spontaneously. However, such droplets with or without encapsulation, rupture following exposure to High Intensity Focused Ultrasound (HIFU). For example, the evaporative rupture of ΔT=8 C superheated liquid filled vesicles whose size is a few microns typically requires ultrasound intensities of 2 MPa [d3]. As mentioned above, treatment which uses such pressures must be localized and requires on-line diagnostics.

It is well known that highly superheated liquid droplets rupture in a process called explosive evaporation. Shusser et al. [d4] developed a model for explosive evaporation by which a vapor filled microbubble is generated within the droplet prior to explosive evaporation. When the microbubble within the volatile liquid reaches a certain radius, the evaporation continues spontaneously until full evaporation. He showed that the critical microbubble radius for spontaneous evaporation r.sub.0 is given by the following equation:

$${r}_{0} = \frac{2 {σ}_{l}}{( {P}_{sl} - {P}_{a} )} \qquad ( 7 )$$

Where P.sub.s1 and u.sub.1 are the volatile liquid vapor pressure and the surface tension, respectively. Using equation (7), and taking into account the properties of PF5050 fluorocarbon (C.sub.5F.sub12) into account, and blood at 39 C show that the critical radius is about 2 micron. Thus, generating r.sub.0=2 micron microbubble within a PF5050 filled vesicle immersed in blood at 39 C would induce spontaneous evaporation of the PF5050 within the vesicle.

Similarly, simultaneous exposure of a vesicle filled with PF5050 mixed with absorbing nanoparticles to electromagnetic radiation and ultrasound would generate a PF5050 vapor filled microbubble within the vesicle. Once the microbubble will grow to the critical radius, the particle content will be evaporated spontaneously followed by its rupture, at much lower ultrasound rarefaction pressure compared to rupture using ultrasound only.

According to preferred embodiments of the present invention there are optionally and preferably provided methods and particles for localized embolization of a blood vessel which overcome the above drawbacks of the background art. The particles are preferably operable for localized generation of a gas bubble in the blood vessels at moderate ultrasound power densities. The gas bubble occludes the blood vessel and in turn abolishes blood flow in it. Occluding the blood flow in multiple blood vessels may be used to treat angiogenesis-dependent diseases including tumor, stop excessive blood loss and block abnormal vessels.

The particle operable for embolization preferably comprises a volatile liquid and absorbing nanoparticles. Exposing the nanoparticles to the combined action of ultrasound and electromagnetic radiation generates a nucleation bubble, which induces rupture in the particle, causing evaporation of the volatile liquid into a gas bubble which occludes the blood vessel.

The particle size is optionally and preferably determined by the content of the low boiling point liquid in the composition, which upon particle rupture, generate the gas bubble intended to occlude the blood vessel. The typical diameter of arterioles and capillaries varies between 25 to 50 micron and 15 to 30 microns, respectively. The typical gas bubble volume is 0.5 to 3 times the blood vessel diameter, cubed.

Effectively occluding a blood vessel requires certain number of gas bubbles which varies according to the blood vessel diameter, length, the gas bubble size and the blood vessel termination structure (e.g., bifurcation) and so forth. Typically, between I and ten gas bubbles are required for effective occlusion of a majority of arterioles and capillaries [d1]. Gas bubbles tend to lodge in bifurcations, and thus block the travel of the remaining bubbles generated within the blood vessel. According to preferred embodiments, between 1 to 10 particles should preferably accumulate on the average in each targeted arteriole or capillary before exposure to ultrasound and electromagnetic radiation.

Within a preferred embodiment, a method for occluding a blood vessel according to the present invention optionally and preferably comprises administering at least one particle comprising volatile liquid suitable for generating a gas bubble to the blood vessel; exposing the vasculature system(s) to simultaneous electromagnetic radiation and ultrasound radiation so as to generate nucleation bubble within the particle; continued exposure of the particle to ultrasound for causing release of the volatile liquid from particle as vapor; and generation of gas bubbles within the blood vessel, such that the blood vessel is effectively occluded.

Preferably, the dominating ultrasound source frequency varies between about 0.1 and about 10 MHz. The dominant frequency may be fixed or scan a range of frequencies continually. Preferably the ultrasound frequency is suitable for therapeutic ultrasound and ranges between about 0.75 and about 3 MHz, with from about 1 and about 2 MHz being more preferred for deep treatments. In terms of mechanical index, the MI optimal for rupture particles for embolization may range between 0.1 and 1.5 and more preferably between 0.3 and 0.8.

In certain aspects, the microwave frequency for rupture particles for embolization may vary between 20 MHz and 10 GHz, and more preferably between 100 MHz and 3 GHz. Each single pulse width is preferably varies between 0.01 and 1000 microsecond. The preferred peak microwave power density range between 0.1 kW/cm2 and 30 kW/cm2. The total dose for a treatment may vary between 1 and 50 J/cm2.

As described above, the composition in the particle may optionally and preferably comprise 0.001 to 1 pico liter and more preferably, between 0.05 and 0.3 Pico liter of low boiling point liquid. In certain aspects of the invention, the particle size optionally varies between 7 and 15 micron and its shape may optionally be cylindrical, spherical or other oblate shape according to flow, controlled rupture and safety requirements.

In some embodiments, the evaporation of volatile liquid is optionally and preferably mitigated to avoid ischemia of the targeted vessel. In preferred embodiment, the particle may comprise a single compartment and single region comprising at least one absorbing nanoparticle and a matrix for holding the nanoparticle. Alternatively the particle may optionally be composed of multiple compartments with at least one absorbing nanoparticle in each one.

The volatile liquid which is evaporated from the particle may optionally and preferably be selected from a group comprising (but not limited to) PF5050 fluorocarbon, 2 fluorobutane 2-methyl cyclobutane octafluoro and other volatile compounds listed by Unger in US Patent application 20050123482. The volatile liquid may also optionally comprise gas precursor, whose boiling temperature is well below 37 C such as perfluorobutane.

In certain aspects the volatile liquid may optionally and preferably comprise absorbing nanoparticles, preferably as clusters. The absorbing nanoparticles may be located on the internal shell surface, or near the center of the particle. The number of absorbing nanoparticles may optionally vary between 1 to 10,000 and more particularly between 30 and 1000. The absorbing nanoparticles may optionally be selected from the choice of absorbing nanoparticles described above. However, they may need minimal functionalization since they are immersed in non-aqueous liquid. The nanoparticles may optionally be coated with suitable material to ensure quick removal by the RES and immune system following rupture of the particle.

According to some embodiments, the particle may optionally and preferably comprise one or more of the following bioactive composition: materials which assist the closure of targeted vasculature such as Endothelin-1, materials which modify the localized blood properties, angiogenic suppression factors comprising anti-invasive factor, such as tissue inhibitor of Metalloproteinase-1, compounds which disrupts microtubule function, such as, for example, paclitaxel, vinblastine, a lighter transition metal (e.g., a vanadium species, which inhibits the formation of new blood vessels), anti-VEGF and additional bioactive composition (see for example Hunter et al in US patent 20060127445), cytoxic compositions effective against cells associated with the angiogenic disease, etc. The bioactive material may optionally be contained in the particle and/or contained within its shell.

In other embodiments the particle may optionally and preferably comprise one or more materials from the following group: absorbing nanoparticles, nanoparticles with ligands which promote attachment to cells or tissue, radioactive material, viruses, emulsion, small size liposomes, etc. Any of these materials may be comprised in the volatile liquid, in separate compartments or integrated into the shell.

The thickness of the particle shell is optionally determined in part by the mechanical properties of the shell, type of material and by the mechanism by which the particle ruptures. The shell thickness will be in the range from about 25 nm to about 1000 nm and more preferably between 200 and 700 nm.

The shell may optionally and preferably comprise absorbing nanoparticles integrated into its structure. In such case, the shell may contain a significant fraction of liquid so as to enable the generation of nucleation. The number of absorbing nanoparticles comprised in the shell may vary between 1 to 3000 and more particularly between 30 and 1000. The absorbing nanoparticles may be selected from the choice of absorbing nanoparticles described above. However, they may need minimal functionalization since they are located in a matrix comprising non-aqueous liquid. The nanoparticles may be coated with suitable material to ensure quick removal by the RES and immune system following rupture of the shell.

In certain aspects, the absorbing nanoparticles which are assigned for generating microbubbles for heating the targeted blood vessels may optionally and preferably be selected from the choice of absorbing nanoparticles described above. Preferably they are functionalized and coated for long circulation period and attached with ligands suitable for attachment to walls of the targeted blood vessels.

The particle may optionally and preferably be administered systemically, by an implant, by injection, or by any other suitable administering procedures. Any standard method can be applied for administering the particles to the targeted vasculature.

### Treatment of Vasculature

According to preferred embodiments, the present invention provides methods for preferably occluding one or more blood vessels which nourish the targeted tissue by generating gas bubble(s) in the blood vessels. The reduced or abolished blood flow to the selected area results in infarction (cell death due to an inadequate supply of oxygen and nutrients) or reduced blood loss from a damaged vessel.

As mentioned above, gas bubble based embolization methods rely on controlled rupture of the particles. The particle should be resistant to accidental rupture either by normal physiological pressures or by the margin of the ultrasound beam. By normal physiological pressures, it is meant those pressures encountered in vivo including pressures within the heart and arteries, as well as compressive pressures of passing through constrictions such as arterioles.

As mentioned above, it is highly desired that the gas bubbles will be generated exclusively in the targeted vasculature. The particles of the present invention may be ruptured locally through (a) attachment of ligands to their external surface as described in U.S. Pat. No. 5,484,584; (b) localized rupture of the particles by HIFU source (see for example Carter at al, in US Patent application 20060052701) operating at moderate ultrasound intensities; (c) Concentrating the particles within the targeted region by the action of external ultrasound energy as described by Dayton et al. in US patent application 20050084538.

The attachment force of typical ligands is much smaller than the blood flow induced drag force on the particles. Thus, targeted particles may drift from their target blood vessels and also accumulate within the vasculature system(s) nourishing healthy tissue, and in turn may ruptured and induce localized ischemia.

The use of HIFU requires mapping of the targeted vasculature. Carter et. al. disclosure describes the identification and location of vasculature system(s) which nourish a diseased tissue as a complicated, slow and costly medical procedure.

As noted above, one way to localize the rupture of particles is by placing two conditions for rupture, each provided by a separate energy source. The present invention provides methods for localizing the particles rupture by either: (a) increasing the composition pressure by using an energy source to deliver energy with limited range within the targeted tissue. (b) Increasing the composition pressure by delivering sufficient energy to a targeted coupling agent. Accordingly, the source energy may be coupled: (a) Directly to the composition within the particle, increasing P.sub.comp or (b) to the blood which surrounds the particles, thus heating the carried composition thereby increasing P.sub.comp.

Within a preferred embodiment, a method for localized treatment of vasculature systems (s) which nourish diseased tissue with particles according to the present invention preferably comprises administering the targeted vasculature system(s) with a mixture containing particles and nanoparticles for the treatment; positioning an ultrasound source and a source of electromagnetic radiation pulses so as to couple their energy to the vasculature system(s); exposing the vasculature system(s) to simultaneous electromagnetic radiation pulses and ultrasound radiation so as to generate microbubbles around the nanoparticles in and at close proximity to the targeted vasculature system; and exposing the vasculature system(s) to the ultrasound radiation so as to heat the blood in the vasculature system to a level sufficient for rupturing the particles, and in turn generation of gas bubbles within the vasculature system(s) such that they are occluded and the blood supply to the diseased tissue is effectively blocked.

In preferred aspects of the present invention, the absorbing nanoparticles comprise antibodies which promote their accumulation on the blood vessel walls of the diseased vasculature. In other aspects, the absorbing nanoparticles are operable for accumulation within the interstitium around the blood vessels, on diseased cells or tissue fed by the targeted vascular system(s) and at a range of up to about one cm around the targeted vascular system(s). In other preferred embodiments, the electromagnetic and ultrasound sources are positioned in accordance with the actual nanoparticles distribution so as to locally heat the blood in the targeted vasculature.

Within another embodiment, a method for localized treatment of tissue with an angiogenesis-dependent disease preferably comprises administering the vasculature system(s) which nourish the diseased tissue so as to accumulate sufficient amount of particles within the targeted vasculature system arterioles; positioning an ultrasound source and a source of electromagnetic radiation pulses so as to couple their energy specifically to the vasculature system(s); exposing the vasculature system(s) to simultaneous electromagnetic pulses and ultrasound radiation so as to generate at least one microbubble within each particle, thus increasing P.sub.comp.; and rupturing particles thereby generating gas bubbles within a majority of the arterioles within the vasculature systems(s) such that they are effectively occluded and in turn, the blood supply to the diseased tissue is effectively stopped.

### Methods for Cancer Treatment

Cancer is the second leading cause of death in the United States, and accounts for over one-fifth of the total mortality. Briefly, cancer is characterized by the uncontrolled division of a population of cells which, most typically, leads to the formation of one or more tumors. Such tumors are also characterized by the ingrowth of vasculature which provides various factors that permit continued tumor growth. Although cancer is generally more readily diagnosed than in the past, many forms, even if detected early, are still incurable.

A variety of methods are presently utilized to treat cancer, including for example, various surgical procedures. If treated with surgery alone however, many patients (particularly those with certain types of cancer, such as breast, brain, colon and hepatic cancer) will experience recurrence of the cancer. Therefore, in addition to surgery, many cancers are also treated with a combination of therapies involving cytotoxic chemotherapeutic drugs (e.g., vincristine) and/or radiation therapy. One difficulty with this approach, however, is that radiotherapeutic and chemotherapeutic agents are toxic to normal tissues, and often create life-threatening side effects. In addition, these approaches often have extremely high failure/remission rates.
Briefly, tumors are typically divided into two classes: benign and malignant. In a benign tumor the cells retain their differentiated features and do not divide in a completely uncontrolled manner. In addition, the tumor is localized and non-metastatic. In a malignant tumor, the cells become undifferentiated, do not respond to the body's growth and hormonal signals, and multiply in an uncontrolled manner; the tumor is invasive and capable of spreading to distant sites (metastasizing).

In many cases, many blood vessels are entering the tumor in all directions similar to the spokes of a wheel. The tumor induces the ingrowth of the host vasculature through the production of “angiogenic factors.” The tumor tissue expands distally along the blood vessels which supply it. Typically, the blood vessel density is greater in the vicinity of the tumor than it is in the surrounding normal tissue. Thus, embolizing the vasculature around a tumor would stop it growth and in turn suffocate it leading to its death.

According to some embodiments of the present invention, a method is optionally and preferably provided for cancer treatment through induction of an embolism as described herein. The method overcomes the drawbacks of the background art, which have had limited success with the use of therapeutic embolization for cancer treatment. Briefly, blood vessels which nourish a tumor are deliberately blocked by injection of an embolic material into the vessels. A variety of materials have been attempted in this regard, including autologous substances such as fat, blood clot, and chopped muscle fragments, as well as artificial materials such as wool, cotton, steel balls, metal coils plastic or glass beads, etc.

Embolization therapy may be utilized in at least three principal ways to assist in the management of neoplasm: (1) definitive treatment of tumors (usually benign); (2) for preoperative embolization; and (3) for palliative embolization. Briefly, benign tumors may sometimes be successfully treated by embolization therapy alone. Examples of such tumors include simple tumors of vascular origin (e.g., haemangiomas), endocrine tumors such as parathyroid adenomas, and benign bone tumors.

For other tumors, (e.g., renal adenocarcinoma), preoperative embolization may be employed hours or days before surgical resection in order to reduce operative blood loss, shorten the duration of the operation, and reduce the risk of dissemination of viable malignant cells by surgical manipulation of the tumor. Many tumors may be successfully embolized preoperatively, e.g., nasopharyngeal tumors.

Embolization may also optionally and preferably be utilized as a primary mode of treatment for inoperable malignancies, in order to extend the survival time of patients with advanced disease. Embolization may produce a marked improvement in the quality of life of patients with malignant tumors by alleviating unpleasant symptoms such as bleeding, venous obstruction and tracheal compression, and humoral effects.

Embolization may optionally and preferably be used as a first line treatment for liver cancer (hepatocellular cancer), although alternatively it may be used a second line treatment or a further treatment.

In one aspect of the present invention, the composition carried by the particle may also optionally comprise an anti-angiogenic factor(s) such as paclitaxol, to complement the treatment by preventing the formation of new blood vessels to supply the tumor or vascular mass by enhancing the devitalizing effect of cutting off the blood supply.

As noted above, embolization therapy utilizing particles of the present invention may also be applied to a variety of other clinical situations where it is desired to occlude blood vessels. Embolization may be accomplished in order to treat conditions of excessive bleeding. For example, menorrhagia (excessive bleeding with menstruation) may be readily treated by embolization of uterine arteries. Arterial embolization may be accomplished in a variety of other conditions, including for example, for acute bleeding, vascular abnormalities, central nervous system disorders, and hypersplenism.

According to preferred embodiments of the present invention, there are optionally and preferably provided compositions and methods for treatment of inflammatory arthritis.

Inflammatory arthritis is a serious health problem in developed countries, particularly given the increasing number of aged individuals. For example, one form of inflammatory arthritis, rheumatoid arthritis (RA) is a multisystem chronic, relapsing, inflammatory disease of unknown cause. Although many organs can be affected, RA is basically a severe form of chronic synovitis that sometimes leads to destruction and ankylosis of affected joints (taken from Robbins Pathological Basis of Disease, by R. S. Cotran, V. Kumar, and S. L. Robbins, W.B. Saunders Co., 1989). Pathologically the disease is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and eventually the pannus grows to fill the joint space.

The pannus develops an extensive network of new blood vessels through the process of angiogenesis which is essential to the evolution of the synovitis. Release of digestive enzymes [matrix metalloproteinases (e.g., collagenase, stromelysin)] and other mediators of the inflammatory process (e.g., hydrogen peroxide, superoxides, lysosomal enzymes, and products of arachadonic acid metabolism) from the cells of the pannus tissue leads to the progressive destruction of the cartilage tissue. The pannus invades the articular cartilage leading to erosions and fragmentation of the cartilage tissue. Eventually there is erosion of the subchondral bone with fibrous ankylosis and ultimately bony ankylosis, of the involved joint.

Thus, within one aspect of the present invention, methods for localized treatment of vasculature systems(s) which nourish the pannus optionally and preferably comprise administering a mixture containing particles and absorbing nanoparticles to the vascular system; positioning an ultrasound source and a source of pulsed electromagnetic radiation so as to couple their energy to the vascular system of the diseased joint; exposing the diseased joint to simultaneous electromagnetic radiation pulses and ultrasound radiation so as to generate microbubbles and in turn heat the blood within the vasculature system; and rupturing the particles in the vasculature system(s) under the combined effect of ultrasound and elevated temperature, and in turn generation of gas bubbles within the vasculature system(s) such that its blood vessels are effectively occluded and blood supply to the pannus is effectively stopped.

In yet another aspect of the present invention, numerous other non-tumorigenic angiogenesis-dependent diseases which are characterized by the abnormal growth of blood vessels may also optionally and preferably be treated by the methods and particles provided by the present invention. Representative examples of such non-tumorigenic angiogenesis-dependent diseases include chronic inflammations and psoriasis.

### Safety Aspects

As noted above, the present invention provides embolization treatment with the following safety features: Particles are not accidentally ruptured in vasculature system(s) nourishing healthy tissue adjacent to the treated vasculature, so as to avoid local ischemia. (b) The generated gas bubbles minimize rupture or damage events within the vasculature system(s) vessels.

As noted above, the particles are ruptured only under the combined action of the ultrasound and electromagnetic radiation for heating the blood or generating a microbubble within each particle. The first method localizes particles rupture within regions wherein nanoparticles has been accumulated. The second method localizes particles rupture due to the limited range of the electromagnetic source radiation within the targeted vasculature region. Thus, the particles and methods provided by the present invention minimize hazards of local ischemia in healthy tissue.

The methods and particles provided by the present invention may provide heating, ultrasound and microbubbles suitable for occluding the arterioles. Occluding the arterioles is beneficial for the present invention in at least two routes: First, it provides sites for lodging traveling gas bubbles. Second, it prevents escape of the gas bubble from the treated region. Thus, the present invention provides methods and particles capable of minimizing possible escape of generated gas bubbles outside the treated region.

In another aspect, the composition within the particle comprises a composition whose pressure P.sub.comp is slightly higher than the normal blood pressure, e.g., 0.12 MPa at 37 C mixed with absorbing nanoparticles of the present invention. Exposure of particles carrying such compositions to the combined action of ultrasound and electromagnetic radiation pulses enable their rupture at reduced P.sub.uls. The expansion dynamics of the generated gas bubble are relaxed compared to particles comprising superheated liquid thereby, reducing the risk of rupture or damage to vasculature system(s) vessel. Thus, the present invention provides embolization methods characterized with reduced damage hazard to the targeted vasculature during the treatment.

As noted above, the methods provided by the present invention increase P.sub.max and thus reducing the ultrasound power density required for particles rupture. Thus, the present invention provides methods enable the use of moderate ultrasound power density, and in turn, eliminating the need for costly on-line diagnostics imaging instrumentation, such as MRI.

### System for Embolization

According some embodiments of the present invention, there is optionally and preferably provided a system for embolization. The system for embolizing a blood vessel provided by the present invention preferably comprises an electromagnetic radiation source, a therapeutic ultrasonic wave generating source and driving means coupled to the therapeutic ultrasonic wave generating source for driving the therapeutic ultrasonic source with a drive signal to generate therapeutic ultrasonic waves.

In some aspects of the present invention, the blood vessel to be treated by the system is first optionally and preferably provided with a mixture of particles and absorbing nanoparticles. Next, the electromagnetic source and the ultrasound source are operated to irradiate the blood vessel during the treatment.

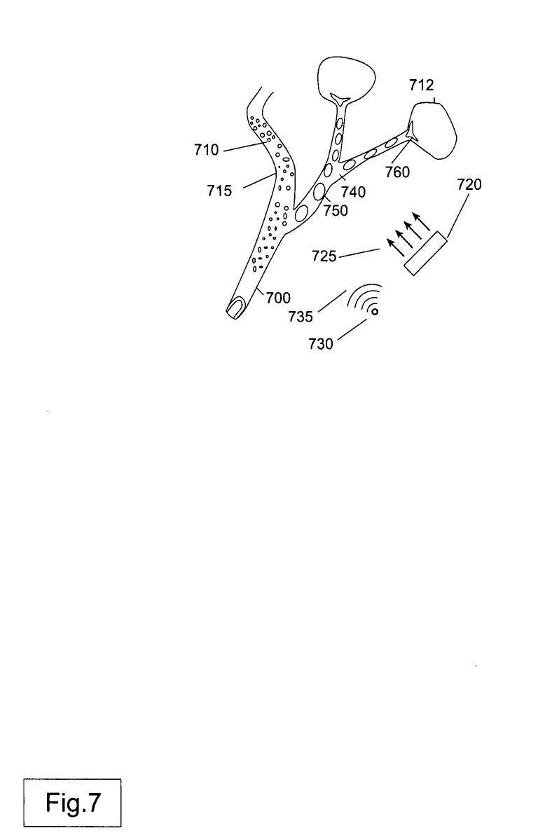

By way of illustration, FIG. 7 shows an exemplary, illustrative but preferred treatment embodiment of vasculature embolization treatment according to the present invention. FIG. 7 shows a detailed view of the vasculature system 700 which feeds a blood vessel 705 which in turn supports angiogenic blood microvasculature 712 within a diseased tissue. The vasculature system 700 is preferably administered with particles 710 suitable for embolizing arterioles and also with absorbing nanoparticles 715, preferably with targeting ligands, operable for attachment to the vasculature system 700 vessels walls, preferably as clusters. An electromagnetic source 720 is operable to expose the vasculature system 700 to a period electromagnetic radiation 725. A suitable ultrasound source 730 is located so as to expose the vasculature system 700 to ultrasound energy 735.

Exposure of particle 710 and nanoparticles 715 to electromagnetic radiation 725 and ultrasound energy 735 induces a microbubble within the particles 710 and free microbubbles 740 on the inner walls of the targeted blood vessel 705. The interaction of the free microbubbles 740 with the ultrasound energy 735 heats the vasculature system 700 region including the blood vessel 705. The heat induced pressure rise within the particle and together with its confined microbubble pulsations rupture the particle 710 and evaporate the carried volatile liquid, thereby creating a bubble 750 which fills the targeted blood vessel 705. The bubble 750 migrates along the blood vessel 705 and may lodge in a bifurcation 760 and occlude the blood vessel 705 and in turn embolize the angiogenic blood microvasculature 712

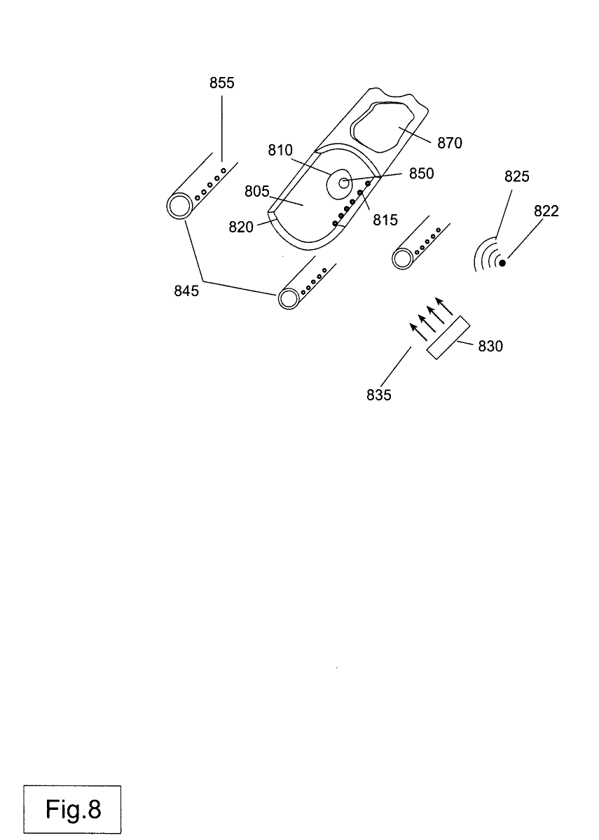

A detailed view of a section of the targeted blood vessel 805 which supports the angiogenic microvasculature during the treatment is illustrated in FIG. 8. At least one particle 810 comprising volatile liquid bioactive composition, and absorbing nanoparticles, is located in the targeted blood vessel 805. Free absorbing nanoparticles 815 which are administered to the blood vessel 805, attach to the internal wall surface 820 of blood vessel 805 as well as within other blood vessels 845.

Simultaneous exposure of particle 810 and nanoparticles 815 to electromagnetic radiation 825 and ultrasound energy 835 induce a microbubble 850 within the particles 810 and free microbubbles 855 on the inner walls of the targeted blood vessel 805. The interaction of the free microbubbles 855 with the ultrasound energy 835 heats the blood in the blood vessel 805 and other blood vessels 845. The blood heating induced pressure rise within the particle 810 and together with its confined microbubble 850 pulsations rupture the particle 810 thereby evaporating the carried volatile liquid, thereby creating a bubble 870 which fills the targeted blood vessel 805. The bubble 870 migrates along the blood vessel 805 and may occlude the blood vessel 805 and in turn, embolize the angiogenic blood microvasculature.

Within another aspect of the present invention, methods for embolizing a blood vessel are provided, optionally and preferably comprising (a) delivering into targeted blood vessel 3 at least one particle 5 carrying composition 50 which includes nanoparticles 10; (b) exposing the blood vessel 3 to simultaneous ultrasound radiation 22 and electromagnetic beam 17 and in turn generating microbubble 24 within the particle 5 (d) rupture of the particle 5 and in turn generating gas bubbles 60 within blood vessel 3 until one of them wedges in the blood vessel 3 and in turn effectively occludes it.

### EXAMPLE 1

Particles comprising 0.1 Pico liter of saturated liquid under 0.12 MPaa at 37 C without voids are provided. The carrying structure is designed for rupture at nominal Pmax=0.1 MPad. The particles are co-administered with nanoparticles as above to the vasculature system of a targeted region. The targeted region is heated according to the procedure described in example 1 to 42 C. At that temperature, the liquid pressure within the particle jumps to 0.165 MPa.sub.a, thereby increasing Pmax to 0.12 MPa.sub.d at the rarefaction phase, resulting in particle rupture probability near 100%

### EXAMPLE 2

Particles comprising 0.1 Pico liter of a liquid whose pressure at 37 C is 0.1 MPa.sub.a and nanoclusters as described above. The liquid comprises a small amount of dissolved fluorocarbon compound whose boiling point is −2 C. The particle carrying structure is designed for rupture at nominal P.sub.max=0.1 MPa.sub.d. The elastic coefficient of the encapsulating material for the carrying structure shell is 70 MPa and the shell thickness is 1 micron. The particle is exposed to the ultrasound and light radiation described above. Under these conditions, at least one microbubble is formed around one nanocluster. In turn, the microbubble grows by rectified diffusion of the volatile fluorocarbon compound, to 1 micron diameter and in turn increases the composition pressure to 0.165 MPa.sub.a. In turn, P.sub.max would reach 0.12 MPa.sub.d resulting in particle rupture probability near 100%.

### EXAMPLE 3

One or more particles comprising 0.1 Pico liter of saturated liquid described in example 1 are administered to an arteriole whose diameter is 36 microns. Nanoparticles described above are co-administered to the region surrounding the arteriole. The region is exposed to the procedure described above, thereby heating the blood in the arteriole to 42 C. Each particle is heated by the surrounding blood to 42 C and rupture as described in example 1 thereby releasing a gas bubble which occupies a cylindrical volume of 36 micron diameter by 75 mm long. One or more gas bubbles are generated in the arteriole, until one of them is wedged in the arteriole. Such a bubble configuration effectively occludes the arteriole and prevents blood flow through it.

### REFERENCES

[d1] T. Ye and J. E. Bull, “Microbubble expansion in a flexible tube,” Trans. ASME v 128 n 8 p. 554-563 (2006).
[d2] G. T. Clement, “Perspectives in clinical uses of high-intensity focused ultrasound,” Ultrasonics v 42 p. 1087-1093 (2004).
[d3] B. Eshpuniyani, J. B. Fowlkes, et. al., “A bench top experimental model of bubble transport in multiple arteriole bifurcations,” Int. J. Heat & Fluid Flow v 26 p. 865-872 (2005).
[d4] M. Shusser, T. Ytrehus, et. al., “Kinetic theory analysis of explosive boiling of a liquid droplet,” Fluid Dynamics Research V 27 p. 35-365 (2000).

### Hyperthermia

Localized heating of cells and tissues is desirable in many therapeutic applications. Precise, localized heating has been shown to have therapeutic benefits, while minimizing the collateral damage to nearby cells and tissue. The therapeutic effects of thermal ablation range from the destruction of cancerous cells and tumors, to the therapeutic or cosmetic removal of benign tumors and other undesirable tissues. Ultrasound energy is the desired heating source due to its simplicity and minimal side effects.

Wu [12] showed that ultrasound energy interaction with microbubbles release heat by while pulsating within aqueous environment. The pulsations are damped by viscous flow in the water while converting the hydrodynamic energy into heat. In preferred embodiments of the present invention, the absorbing nanoparticles loading, and ultrasound source operation conditions are selected so that the energy coupled to the microbubbles release heat.

The present invention encompasses the use of absorbing nanoparticles for localized heating of patient body regions by their interaction with microwave radiation, evolution of microbubbles, followed by interaction of the induced microbubbles with ultrasound radiation.

In a particular application of the invention, the treatment is based on release of heat from ultrasound interaction with multiple microbubbles generated near cells, tissue or a non-tissue material and comprises: a) administering absorbing nanoparticles to the targeted cells tissue or non tissue material; (b) positioning an ultrasound source and a source of microwave radiation so as to couple their energy to the cell or tissue (c) Further exposing the cells or tissue to simultaneous microwave radiation and ultrasound radiation so as to generate microbubbles around the nanoparticles in and at close proximity to the targeted cells or tissue (d) Exposing the cells or tissue to the ultrasound radiation so as to release heat the cells, tissue or non tissue material.

In one embodiment of the invention, the hyperthermia treatment takes advantage of the enhanced nanoparticles transport through the vascular system(s) at elevated temperatures. Kong, et al., Cancer Res., 2001, 61, 3027 teach that hyperthermia accelerates the passage of nanoparticles through the capillaries of the vascular system of growing tumors. Hyperthermia will also enhance the uptake of the absorbing nanoparticles within other types of diseased tissue, such as sites of inflammation caused by infection or trauma.

### Section 5—Applications of Microbubbles for Imaging Diagnostics

According to preferred embodiments of the present invention there is provided a method for using microbubbles (see for example Barak et al. [e1]) or nanobubbles, generated according to the present invention, for imaging diagnostics. When a small group of nucleation bubbles is generated near absorbing nanoparticles within a defined liquid volume, they could be employed for diagnostic imaging using ultrasound transducers operating at the tens MHz range [e2]. Attachment of ligand molecules to these absorbing nanoparticles enables diagnostic imaging of the targeted cells tissue or non-tissue material.

When these nucleation bubbles evolve into microbubbles their presence and location may be employed for ultrasound diagnostics imaging operating at MHz range similar to contrast agent bubbles. Thus, nucleation bubbles generated by exposing absorbing nanoparticles to microwave radiation and their subsequent evolution into microbubbles can be used as well for diagnostic imaging.

Diagnostic imaging of the biofilm contaminated tissue is important for navigating the treatment region within the patient region and for diagnosing the diseased tissue status during the course of the treatment and during follow-up procedures. For example, administering targeted absorbing nanoparticles to contaminated tissue may induce multiple groups of microbubbles (=bright spots) in the ultrasound image due to the presence of biofilm in the imaged tissue. Diagnostic imaging may also be used to identify bacterial contaminations in implants and non-medical surfaces.

A preferred embodiment of the present invention provides a method for imaging diagnostics of at least one biofilm comprising administering absorbing nanoparticles to the biofilm(s); positioning at least one ultrasound source and at least one source of electromagnetic radiation so as to couple ultrasound radiation and electromagnetic radiation to the biofilm(s); exposing the biofilm to simultaneous electromagnetic radiation pulses and ultrasound radiation thereby generating a microbubble around one or more nanoparticles on the biofilm for diagnostic imaging of the biofilm.

The ability to provide imaging diagnostics during localized drug delivery has several applications. One example is cancer therapy, wherein toxic drugs are used to treat the tumor. Without on-line imaging diagnostics, localized release of drug outside the tumor may damage healthy tissue. As described above, the nanobubbles generated by exposure of absorbing nanoparticles to microwave radiation are sufficient for providing imaging diagnostics data.

In certain aspects of the invention, the nanoparticles are used to perform angiography (a road map of the blood vessels) prior to embolization treatment of the desired vasculature(s). The vasculature system is exposed to the combined effect of ultrasound and electromagnetic radiation which for generating microbubble cloud sufficient for diagnostic imaging. Next, the desired vasculature system(s) is than embolized according to the angiography data, using the particles and methods provided by the present invention.

In another aspect, the diagnostics imaging data of the particles ultrasound images together with the ultrasound images of microbubbles is combined for angiography of the targeted vasculature system(s). In other aspects, diagnostic imaging which can identify the particles motion is used to assess the effectiveness of the embolization treatment. In yet another aspect, ultrasound images of the particles is used to control a localized drug release procedure based on rupture of particles suitable for the present invention.

In certain aspects, the ultrasound and electromagnetic radiation intensities used for imaging diagnostics are similar to those used for the previously described applications of the present invention. This feature may be useful for on-line imaging diagnostics. In other aspects the ultrasound and electromagnetic radiation intensities are significantly lower compared to those required for previously described applications. This feature may be useful for acquire imaging information on the targeted region and the distribution of the absorbing nanoparticles within the region before conducting the treatment. In yet other aspects, the ultrasound and/or electromagnetic radiation intensities are modulated in a different sequence than those used for the actual application. This mode of operation may be useful for enhancing the sensitivity of the diagnostic imaging.

In a preferred embodiment, the absorbing nanoparticles suitable for imaging diagnostics are similar to those used for the specific application. In other aspects the absorbing nanoparticles are optimized for generating nucleation bubbles at lower ultrasound and electromagnetic radiation intensities. Such feature may be useful for mapping the absorbing nanoparticles distribution within the region of patient.

In preferred embodiments, the particles of the present invention operable for imaging diagnostics during localized drug delivery are similar to those used for drug delivery. In other aspects they are optimized for maximum interaction cross section with the ultrasound energy. This feature may be useful for obtaining diagnostic image from small amount of particles, a small region of targeted cells or tissue, or in hard-to-access regions of patient. In other aspects they are optimized for internal generation of nucleation bubbles at lower ultrasound and electromagnetic radiation. Such feature may be important to map the distribution of the particles within the region of patient before conducting localized drug delivery at higher ultrasound and electromagnetic radiation intensities.
[e1] M. Barak and Y. Katz, “Microbubbles: Pathophysiology and Clinical Implications,” Chest V 128 p. 2918-2932 (2005).
[e2] M. Odonnell, L. Balogh et al, “Colloid loaded dendrimers for fighting cancer,” Nanotech Conf. May 24, 2007.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for generating a nucleation bubble in a non-thermal process, comprising: Providing a nanoparticle in a liquid environment; and Applying electromagnetic radiation to said nanoparticle to induce formation of a nucleation bubble.

2. The method of claim 1, wherein said electromagnetic radiation comprises microwave radiation.

3. The method of claim 1, wherein said nanoparticle induces local electromagnetic radiation whose electric field magnitude is at least five times ambient electromagnetic field.

4. The method of claim 1, further comprising applying ultrasound to said nanoparticles.

5. The method of claim 4, wherein said ultrasound is applied to grow said nucleation bubble to form a microbubble.

6. The method of claim 1, wherein said electromagnetic radiation comprises microwave radiation of a frequency from about 20 MHz to about 1000 GHz.

7. (canceled)

8. The method of claim 6, wherein a source pulse width of said microwave radiation is from about 10 nanosecond to about 30 milliseconds.

9. The method of claim 8, wherein said source pulse width is from about 0.01 to about 10 microsecond.

10. The method of claim 6, wherein an average microwave power density is from about 0.1 kW/cm2 to about 1 MW/cm2.

11-12. (canceled)

13. The method of claim 6, further comprising applying ultrasound having an ultrasound source frequency of from about 20 kHz to about 10 MHz.

14-15. (canceled)

16. The method of claim 13, wherein an energy level of said ultrasound is from about 0.5 Watt (W) per square centimeter (cm.sup.2) to about 20 W/cm.sup.2.

17. (canceled)

18. The method of claim 13, further comprising synchronizing applying said ultrasound radiation and said microwave radiation.

19. The method of claim 1, further comprising providing nanoparticles to an object to be treated.

20-21. (canceled)

22. The method of claim 1, wherein said nanoparticles comprise conductive material in the microwave frequencies.

23-25. (canceled)

26. The method of claim 1, wherein a shape of said nanoparticle is selected from the group consisting of nanotubes, high aspect ratio rods or ellipsoids, and nanoshells.

27-29. (canceled)

30. The method of claim 1, wherein said nanoparticle comprises at least one site for promoting the accumulation of gas molecules generated by exposing said nanoparticle to microwave radiation.

31-32. (canceled)

33. A method for generating a microbubble in a non-thermal process, comprising:
Providing a nanoparticle in a liquid environment; Applying electromagnetic radiation to said nanoparticle to induce formation of a gas nucleation bubble; and Applying ultrasound to form a microbubble from said gas nucleation bubble.

34. (canceled)

35. A method for generating a microbubble, comprising:
Providing a nanoparticle in a liquid environment;
Applying electromagnetic radiation to said nanoparticle to induce formation of reactive species molecules;
Generating a gas nucleation bubble from a reaction of said reactive species and said liquid environment; and
Forming a microbubble from said gas nucleation bubble.

36. A method for generating a microbubble, comprising:
Providing a nanoparticle in a liquid environment;
Applying microwave radiation to said nanoparticle to induce formation of a gas nucleation bubble;
Applying ultrasound radiation to said gas nucleation bubble to form a microbubble; and
Increasing a size of said microbubble through continued application of said ultrasound radiation.

37-38. (canceled)

39. A composition for inducing formation of a microbubble upon application of a non-thermal process, comprising a nanoparticle having a surface featuring at least one characteristic for accumulation of gas molecules, wherein said gas molecules form a nucleation seed for the microbubble.

40-57. (canceled)

58. A system for inducing a microbubble in a non-thermal process, comprising:
a. a source of microwave radiation;
b. a source of ultrasound radiation;
c. a guide for said microwave radiation and said ultrasound radiation; and
d. a nanoparticle in a liquid environment for receiving said microwave radiation and said ultrasound radiation, and for generating the microbubble.

59. A method for biofilm treatment, comprising: generating a microbubble in a non-thermal process according to claim 1.

60-67. (canceled)

68. The method of claim 59, wherein said absorbing nanoparticles are arranged in clusters and the clusters comprise between 5 and 50 nanoparticles each.

69. The method of claim 59, wherein an average inter-nanoparticle distance ranges from about 0.1 to about 3 microns.

70-73. (canceled)

74. A composition for delivery of a bioactive agent comprising:
a particle comprising a bioactive composition, volatile liquid, and absorbing nanoparticles operable for inducing delivery of the bioactive agent when exposed to suitable electromagnetic and ultrasound radiation.

75-83. (canceled)

83. A method for localized delivery of a bioactive composition from a particle, comprising:
delivering a particle comprising bioactive composition, absorbing nanoparticles and volatile composition to cells or tissue; and
exposing said particle to simultaneous electromagnetic radiation beam and ultrasound radiation to induce release of the bioactive composition.

84. The method of claim 83, further comprising:
generating a microbubble within said particle sufficient to evaporate at least a fraction of said volatile composition; and
breaching said particle due to enhanced internal pressure, thereby causing release of its bioactive content to said cells or tissue.

85. (canceled)

86. A method for localized delivery of therapeutic or bioactive composition from a particle, comprising:
delivering a particle comprising bioactive composition, absorbing nanoparticles and pro-permeable membrane wall and an attached ligand suitable for attachment to a targeted cell, to the eye;
contacting said particle to selected ocular target cells using a suitable ligand; and
exposing said particle simultaneous electromagnetic radiation beam and ultrasound radiation.

87. The method of claim 86, further comprising:
generating a microbubble near inner wall of said particle; and
inducing permeability of the membrane shell due to pulsation of said microbubble, in turn enabling enhanced transport of said bioactive compositions from said particle to said targeted cells or tissue.

88-96. (canceled)
